(19) United States
(12) Patent Application Publication
(10) Pub. No.: US 2015/0064141 A1
(43) Pub. Date: Mar. 5, 2015

(54) REGENERATIVE SERA CELLS AND MESENCHYMAL STEM CELLS

(71) Applicant: The Regents of the University of California, Oakland, CA (US)

(72) Inventor: James A. Byrne, Santa Monica, CA (US)

(21) Appl. No.: 14/390,738

(22) PCT Filed: Mar. 14, 2013
(86) PCT No.: PCT/US2013/031517

§ 371 Date: Oct. 3, 2014

Related U.S. Application Data
(60) Provisional application No. 61/620,837, filed on Apr. 5, 2012.
(60) Provisional application No. 61/692,063, filed on Aug. 22, 2012.

(51) Int. Cl. A61K 35/28; G01N 33/569

(52) U.S. Cl. CPC A61K 35/28; G01N 33/56966; G01N 2333/70596
USPC 424/93.7; 435/366; 435/7.21

(57) ABSTRACT

Disclosed herein are three types of skin-derived cells. One type of the cells is mesenchymal stem cells characterized by expression of the cell surface biomarker cluster of differentiation (CD) 146, the second type expresses CD271. The third type of cells is regeneration-associated cells characterized by expression of the cell surface biomarkers stage-specific embryonic antigen 3 (SSEA3) and CD 105 (clone 35). Also disclosed are methods of isolating, purifying, culturing, storing, and using these cells.

### CROSS-REFERENCE TO RELATED APPLICATIONS

This application claims benefit of and priority to U.S. Ser. No. 61/692,063, filed Aug. 22, 2012, and to U.S. Ser. No. 61/620,837, filed Apr. 5, 2012, both of which are incorporated herein by reference in their entirety for all purposes.

### STATEMENT OF GOVERNMENTAL SUPPORT

This work was supported in part by Grant No: RC2 HK103400 from the National Institutes of Health (NIH). The Government has certain rights in this invention.

### BACKGROUND

There is a need for ways to enhance, restore, and maintain organ function. Stem cells hold much promise for accomplishing the goals of regenerative medicine. Human embryonic stem (ES) cells, induced pluripotent stem (iPS) cells, and mesenchymal stem cells (MSCs) have all been advanced as sources for tissue engineering and regenerative medicine (Badylak and Nerem (2010) Proc. Natl. Acad. Sci. USA, 107(8): 3285-3286). MSCs, the newest class of these stem cells, have emerged as the most promising. Mesenchymal stem cells are a class of pluripotent cells, found in various tissues, that can give rise to a number of different tissue cell types including, such as osteocytes, chondrocytes, adipocytes, endothelial cells, fibroblasts, and smooth muscle cells. Advantages of MSCs include their use for autologous treatment and their relative lack of negative side effects (Trounson (2009) BMC Med, 7(1): 29).

Mesenchymal stem cells have been isolated and purified from different sources such as bone marrow (Kern et al. (2006) Stem Cells 24(5): 1294-1301; Friedenstein et al. (1976) Exp. Hematol. 4: 276), adipose tissue (Kern et al. 2006), umbilical cord blood (Kern et al. 2006), placenta (Ilic et al. (2011) Meth. Mol. Biol., 698: 89-106), umbilical cord matrix (Cai et al. (2010) J. Biol. Chem., 285(15): 11227-11234), amniotic membrane (Cai et al. 2010), and dental pulp (Marchionni et al. (2009) Int. J. Immunopathol. Pharmacol., 22(3): 699-706) and have been found in the stroma of various tissues and organs. Enriched populations of mesenchymal stem cells (from bone marrow cells) can be induced, by choice of culture conditions, to differentiate into various types of connective tissue, including bone, cartilage and adipose tissue (Prockop (1997) Science 276: 71-74; Pittenger et al. (1999) Science 284: 143-147). Mesenchymal stem cells have also been isolated from skin dermis (Park et al. (2012) Differentiation 83: 249-259; Shariullina et al. (2007) Eur. Cells and Materials 13, Suppl. 2: 76; Salvolini et al. (2010) Exp. Dermatology, 19: 848-850; Riekstina et al. (2008) Cytotechnology 58: 153-162; Haniffa et al. (2007) J Immunol., 179(3): 1595-1604; Shih et al. (2005) Stem Cells, 23(7): 1012-1020; Chen et al. (2007) J. Cell Sci., 120(Pt 16): 2875-2883; Shi and Cheng (2004) World J. Gastroenterol., 10(17): 2550-2552; Bi et al. (2010) BMC Cell Biol., 11: 46; Lorenz et al. (2008) Exp. Dermatol., 17(11): 925-932; Crigler et al. (2007) FASEB J., 21(9): 2050-2063; Bartsch et al. (2005) Stem Cells Dev. 14(3): 337-348).

It is estimated that eight million people per year in the United States suffer from wounds caused by mechanical trauma, vascular insufficiencies or diabetes and if these wounds are left untreated, death due to infection can occur. An incision created by a surgeon, trauma as a result of blunt force, or tissue death caused by a variety of diseases all undergo a similar process of wound healing. Wound healing occurs in three distinct phases. The inflammatory phase is characterized by inflammation at the site of the trauma. This phase is critical for healing and involves extensive cell migration. The second phase of wound healing is the proliferative phase, which is marked by epithelialization, angiogenesis, granulation tissue formation and collagen deposition. The third and final stage of wound healing is the maturational phase where fibroblasts differentiate into collagen. The disposition of the connective tissue matrix and collagen undergoes a contraction, resulting in scar tissue. Although scar formation is important to wound healing, excessive scar formation can have additional cosmetic and/or pathologic consequences, such as keloids and/or hypotrophic scars. Scar formation can occur in all tissues and the adverse effects of scar formation include keloid, hypertrophic scars, burn contracture and scleroderma in skin. The ability of a wound to heal with minimal scar formation can have a profound effect on the patient and on medical or surgical practice.

Although numerous materials and techniques are used to treat skin wounds and to restore and regenerate skin tissue, the cells and processes responsible for initiation and promotion of wound healing in skin have not been identified and are not well understood.

### SUMMARY

Skin-derived cells that represent two rare and distinct populations of cells in primary mammalian skin cell cultures are isolated, purified, cultured, stored, and used. Cells from the two populations, and cells derived from them, have distinct properties and uses. The first skin-derived cell population is characterized by expression of the cell surface biomarker cluster of differentiation (CD) 271. Cells in this population are highly enriched mesenchymal stem cells and can be used for any use for stem cells or mesenchymal stem cells. The second skin-derived cell population is characterized by expression of the cell surface biomarkers stage-specific embryonic antigen 3 (SSEA3) and CD105 (clone 35). Cells in this population are highly enriched skin regeneration cells and exhibit a significant proliferative response advantageous in skin repair and regeneration.

Human cells that are derived from the dermis of human skin (or other mammalian skin) are isolated. The cells can be mesenchymal stem cells (MSCs) and express CD146 and CD271. These cells are referred to as CD146-MSCs and CD271-MSCs, respectively. The cells can express stage-specific embryonic antigen 3 (SSEA3) and can bind anti-human CD105 antibody clone 35. These cells can be referred to as SERA cells.

CD146-MSCs and CD271-MSCs can be substantially isolated from human dermal skin cells that were present when the cells were derived and that do not significantly express CD146 or CD271. CD146-MSCs and CD271-MSCs can differentiate into adipocytes, osteoblasts, or chondrocytes under conditions that induce such differentiation. CD146-MSCs and CD271-MSCs can express one or markers selected from the group consisting of CD10, CD13, CD26, CD29, CD34, CD44, CD54, CD71, CD73, CD90, CD105 (clone 266), CD106, CD166, ITGA11, STRO-1 and SSEA4. CD146-MSCs and CD271-MSCs are useful to generate differentiated cells and for producing induced pluripotent stem cells (iPSCs).

SERA cells can be substantially isolated from human dermal skin cells that were present when the cell was derived, that expressed SSEA3, and that did not significantly bind CD105 antibody clone 35. SERA cells can express a high level of SSEA3. SERA cells generally do not differentiate into adipocytes, osteoblasts, or chondrocytes, under conditions that induce such differentiation. SERA cells can participate in wound repair following injury. SERA cells are useful for tissue repair and regeneration and for producing iPSCs.

SERA cells, CD146-MSCs and/or CD271-MSCs can be isolated from an in vitro culture of primary human skin cells or derived from adult human skin.

CD146-MSCs and CD271-MSCs can be differentiated to various types of cells, such as osteoblasts, osteocytes, chondroblasts, chondrocytes, cardiomyoblasts, cardiomyocytes, neuroblasts, neurocytes, pancreocytes, hepatoblasts, hepatocytes, and hematopoietic stem cells. Such differentiated cells can be used for therapeutic, research, and other purposes.

Also disclosed are cell lines and cell strains that include a plurality of cells proliferated ex vivo from the CD146-MSCs, CD271-MSCs or SERA cells. Also disclosed are cell lines and cell strains that include a plurality of cells differentiated ex vivo from the CD146-MSCs, CD271-MSCs or SERA cells. Also disclosed are cell lines and cell strains that include a plurality of induced pluripotent stem cells (iPSCs) derived ex vivo from the CD271-MSCs or SERA cells. Also disclosed are cell lines and cell strains that include a plurality of cell differentiated ex vivo from iPSCs derived from the CD146-MSCs, CD271-MSCs or SERA cells.

Also disclosed are therapeutic compositions that include (a) SERA cells, CD146-MSCs, CD271-MSCs, and/or cells derived from the SERA cells, CD146-MSCs, CD271-MSCs, or all three and (b) a pharmaceutically acceptable platform. The cells can be derived by proliferation, differentiation, induction, etc. from the CD146-MSCs, CD271-MSCs or SERA cells. The platform can be a graft. The graft can include a scaffold and a therapeutic component. The scaffold can include a porous matrix of fibers forming interstices. The therapeutic component can include cellular components and/or non-cellular components. The cellular component can include the SERA cells, CD146-MSCs and CD271-MSCs, and/or cells derived from the SERA cells, CD146-MSCs, CD271-MSCs, or all three. The non-cellular component can include one or more therapeutic, prophylactic, or diagnostic agents such as growth factors, anti-inflammatories, antibiotics, and antivirals.

Also disclosed are methods of using SERA cells, CD146-MSCs, CD271-MSCs, and cells derived from the SERA cells, CD146-MSCs and CD271-MSCs. Methods of treating a mammal can involve applying or administering to the mammal SERA cells, CD146-MSC, CD271-MSCs, and/or cells derived from the SERA cells, CD146-MSCs, CD271-MSCs, or all three. For example, the SERA cells, CD146-MSCs, CD271-MSCs, and/or cells derived from the SERA cells, CD146-MSCs, CD271-MSCs, or all three can be administered to a patient by way of a composition that includes the cells alone or on or in a carrier or support structure. In many cases, no carrier will be required. In these cases, the cells typically have been washed to remove cell culture media and will be suspended in a physiological buffer. The cells can be administered alone or in combination with other pharmaceutical or bioactive agents. The cells can be used in grafts, implants, tissue engineering compositions, and in devices for tissue repair, regeneration, augmentation, and engineering.

Also disclosed are methods of producing isolated CD146-MSCs and CD271-MSCs. Cells from a tissue sample including dermis of human skin (or other mammalian skin) can be cultured and a subpopulation of the cultured cells that express CD146 or CD271 isolated. Also disclosed are methods of producing isolated SERA cells, by culturing cells from a tissue sample including dermis of human skin (or other mammalian skin), and isolating a subpopulation of the cultured cells that express high levels of SSEA3 and that are bound by anti-human CD105 antibody clone 35. The subpopulations can be cultured ex vivo.

Skin-cell derived mesenchymal stem cells characterized by expression of the cell surface biomarker CD146 and CD271 can be more easily obtained, stored, cultured, expanded, and/or differentiated than other multipotent cells due, it is believed, to the convenience of their isolation and their relative purity. CD146-MSCs and CD271-MSCs can be derived from the dermis of human skin (or other mammalian skin) by selecting, sorting, or enriching for cells expressing CD146 and/or CD271. CD146-MSCs and CD271-MSCs can be produced by obtaining a tissue sample from dermis of human skin (or other mammalian skin); culturing cells from the sample; and isolating a subpopulation of the cells that expresses CD146 or CD271, respectively. Thus, CD146-MSCs and CD271-MSCs can be isolated from an in vitro culture of primary human skin cells. CD146-MSCs and CD271-MSCs can be cultured and stored ex vivo.

The CD146-MSCs and CD271-MSCs can be used for any purpose and in any way that MSCs can be used. For example, CD146-MSCs and CD271-MSCs can be cultured and then differentiated to various types of multipotent mesenchymal stem cells, such as human osteogenic dermal mesenchymal stem cells (HOD-MSCs), human chondrogenic dermal mesenchymal stem cells (HCD-MSCs), and human adipogenic dermal mesenchymal stem cells (HAD-MSCs). Such differentiated MSCs can be used for therapeutic, research, and other purposes. Thus, provided herein are methods of treatment using differentiated MSCs and cells differentiated from differentiated MSCs.

Also disclosed herein are skin-cell derived SSEA3-expressing regeneration-associated (SERA) cells characterized by expression of the cell surface biomarkers SSEA3 and CD105 (clone 35). SERA cells can be derived from the dermis of human skin (or other mammalian skin) by selecting, sorting, or enriching for cells expressing SSEA3 and bound by anti-human CD105 antibody clone 35. Methods of producing SERA cells can involve, for example, obtaining a tissue sample from dermis of human skin (or other mammalian skin); culturing cells from the sample; and isolating a subpopulation of the cells that expresses high levels of SSEA3 and that are bound by anti-human CD105 antibody clone 35. Thus, SERA cells can be isolated from an in vitro culture of primary human skin cells. SERA cells can be cultured and stored ex vivo. SERA cells can be used for a variety of therapeutic, research, and other purposes. SERA cells are associated with regeneration of skin tissue. Thus, disclosed are methods of treatment using SERA cells and cells derived from SERA cells. In particular, SERA cells can be used to aid in skin growth, repair, and regeneration. For example, SERA cells can be cultured and introduced to subjects in need of skin repair or regeneration. Also disclosed are cell lines and cell strains generated from proliferation of SERA cells ex vivo from skin dermis.

SERA cells can have high expression of SSEA3 (SSEA3^{HIGH}).

SERA cells, CD146-MSCs and CD271-MSCs can be grown, stored and otherwise maintained over a number of passages. The cells can be high passage cells, with Passage (P) equal or greater than 8. The cells can be low passage cells with P<8, or P<7, or P<6, or P<5, or P<4. The cells can be maintained in culture for less than about 6 weeks, or for less than about 1 month, or for less than about 2 weeks, or for about 1 week or less. The cells can be derived from adult human skin.

CD146-MSCs, CD271-MSCs and SERA cells can be used to produce induced pluripotent stem cells (iPSCs). Such iPSCs can be used in place of pluripotent stem cells, such as embryonic stem cells (ESCs), MSCs, marrow adherent stromal cells (MAPCs), MIAMI, hematopoietic stem cells (HSCs), and especially uses particularly suited to iPSCs, such as for autologous treatments.

Also disclosed are devices that include cells derived from SERA cells, CD146-MSCs and/or CD271-MSCs. Useful devices include implants, rods, pins, screws, braces, plates, prosthesis, tissue engineering scaffolds, and/or grafts. Thus, for example, disclosed are devices or grafts having a scaffold and a therapeutic component. The scaffold can be, for example, a porous matrix of fibers forming interstices and the therapeutic component can have, for example, a cellular component and a non-cellular component. The cellular component can be, for example, SERA cells, CD146-MSCs, CD271-MSCs, and/or cells derived from the SERA cells, CD146-MSCs, CD271-MSCs, or all three. The fibers can be synthetic fibers. The fibers can be biodegradable polymeric fibers. The device or graft can have fibers derived from a biomaterial. The graft can have collagen fibers. The device or graft can have an artificial skin material. The non-cellular component can have cell culture medium and/or synthetic or natural extracellular matrix (ECM). The non-cellular component can have one or more pharmaceutically active agents. For example, the pharmaceutically active agents can be one or more growth factors, anti-inflammatories, antibiotics, antivirals, or a combination.

Also disclosed are skin grafts where the grafts have skin or a material derived from skin where the skin or material derived from skin is impregnated with SERA cells, CD146-MSCs, CD271-MSCs, and/or cells derived from the SERA cells, CD146-MSCs, CD271-MSCs, or both.

Also disclosed are methods of treating a wound in a mammal. The methods can involve applying or administering to a subject SERA cells, CD146-MSCs, CD271-MSCs, cells derived from the SERA cells, CD146-MSCs, CD271-MSCs, or both, and/or a device or graft having SERA cells, CD146-MSCs, CD271-MSCs, and/or cells derived from the SERA cells, CD146-MSCs, CD271-MSCs, or both. The wound can be a burn wound, a laceration or abrasion, a radiation-induced wound, a diabetes related chronic wound, a surgical wound, or a combination. The SERA cells, CD146-MSCs and CD271-MSCs can be autologous or heterologous.

Accordingly, in various embodiments, skin-cell derived mesenchymal stem cells are provided that can be more easily obtained, stored, cultured, expanded, and differentiated. In addition, methods for generating and using such skin-cell derived mesenchymal stem cells are also provided. Similarly, in certain embodiments, methods for generating and using differentiated multipotent mesenchymal stem cells from such skin-cell derived mesenchymal stem cells are provided. In certain embodiments methods for generating and using differentiated cells from such skin-cell derived mesenchymal stem cells are provided. In certain embodiments skin-cell derived regeneration-associated cells are provided as well as methods for generating and using such skin-cell derived regeneration-associated cells. In certain embodiments methods for using such skin-cell derived regeneration-associated cells to aid in skin growth, repair, and regeneration are provided. In certain embodiments methods for generating and using induced pluripotent stem cells from such skin-cell derived mesenchymal stem cells and regeneration-associated cells are provided.

### BRIEF DESCRIPTION OF THE DRAWINGS

The accompanying drawings, which are incorporated in and constitute a part of this specification, illustrate several embodiments of the disclosed cells, methods, and compositions, and, together with the description, serve to explain the principles of the disclosed cells, methods, and compositions.

FIGS. 1A and 1B show global transcriptional meta-analysis of SSEA3 expressing human adult skin-derived cells. FIG. 1A: Relative gene expression analysis in comparison to SSEA3^{NEGATIVE} cells. FIG. 1B: Unbiased whole-transcriptome cluster analysis and representative sample of transcriptome comparison heatmap. * Gene expression ratio calculated based on the average gene expression correlation (set at 0.0) compared to the most similar identified cell type (set at 1.0).

FIGS. 2A-2D illustrate the dynamics of SSEA3 expression during in vitro culture. FIG. 2A: Flow cytometry analysis of CD105 antibody clones 266 and 35 in skin biopsy derived human cells. FIG. 2B: Flow cytometry analysis of SSEA3 and CD105 clone 35 expression in skin biopsy derived cells. FIG. 2C: Fluorescence activated cell sorting (FACS) based analysis and purification of SSEA3^{NEGATIVE} (low or undetectable expression) and SSEA3^{HIGH} (high expression) subpopulations and analysis of these subpopulations 1 day and 1 week after cell sorting. FIG. 2D: FACS analysis of SSEA3^{HIGH} cells purified from higher passage (p8) and lower passage (p3) cells 1, 3 and 7 days after cell sorting.

### DETAILED DESCRIPTION

### I. Definitions

“Mesenchymal stem cell” or “MSC” refer to multipotent stem cells present in or derived from mesenchymal tissue that can differentiate into a variety of cell types, including: osteoblasts, chondrocytes, and adipocytes.

“Cell” refers to individual cells, cell lines, primary cultures, or cultures derived from such cells unless specifically indicated. “Culture” refers to a composition including isolated cells of the same or a different type. “Cell line” refers to a permanently established cell culture that will proliferate indefinitely given appropriate fresh medium and space, thus making the cell line “immortal.” “Cell strain” refers to a cell culture having a plurality of cells adapted to culture, but with finite division potential. “Cell culture” is a population of cells grown on a medium such as agar.

The terms “primary cells”, “primary cell lines”, and “primary cultures” are used interchangeably herein to refer to cells and cells cultures that have been derived from a subject and allowed to grow in vitro for a limited number of passages, that is, splittings, of the culture. For example primary cultures are cultures that have been passaged 0 times, 1 time, 2 times, 4 times, 5 times, 10 times, or 15 times, but not enough times go through the crisis stage. Typically, the primary cell lines can be maintained for fewer than 10 passages in vitro. “Primary skin cell culture” refers to a primary cell culture derived from skin cells. A cell can be in vitro or ex vivo. Alternatively, a cell can be in vivo and can be found in a subject. A cell can be a cell from any organism including, but not limited to, animals.

The terms “differentiated somatic cell” or simply “somatic cell” encompass any cell in or of an organism that cannot give rise to all types of cells in an organism. In other words, somatic cells are cells that have differentiated sufficiently that they will not naturally generate cells of all three germ layers of the body, that is, ectoderm, mesoderm and endoderm. For example, somatic cells would include both neurons and neural progenitors, the latter of which are able to naturally give rise to all or some cell types of the central nervous system but cannot give rise to cells of the mesoderm or endoderm lineages.

“Isolated,” “isolating,” “purified,” “purifying,” “enriched,” and “enriching,” when used with respect to cells, indicate that the cells at some point in time were separated, enriched, sorted, differentially proliferated, etc., from or with respect to other cells resulting in a higher proportion of the cells compared to the other cells. “Highly purified,” “highly enriched,” and “highly isolated,” when used with respect to cells, indicates that the cells of interest are at least about 70%, about 75%, about 80%, about 85% about 90% or more of the cells, about 95% or more of the cells, and can preferably be about 95% or more of the cells. “Substantially isolated,” “substantially purified,” and “substantially enriched,” when used with respect to cells, indicate that the cells of interest are at least about 70%, about 75%, or about 80% of the cells, more usually at least 85% or 90% of the cells, and sometimes at least 95% or more of the cells, for example, 95%, 96%, and up to 100% of the cells.

“Population,” when used with respect to cells, refers to a group or collection of cells that share one or more characteristics. For example, skin cells that express SSEA3 and CD105 (clone 35) can be said to be a population of skin cells characterized by expression of these markers. The term “subpopulation,” when used with respect to cells, refers to a population of cells that are only a portion or subset of a population of cells. For example a set of skin cells that express SSEA3 and CD105 (clone 35) can be said to be a subpopulation of skin cells that express SSEA3.

“Passaging” and “passage,” when used with respect to cells, refer to replacing the culture media or transferring cells to new culture media. “Skin-derived cell” refers to cells isolated from skin tissue and cells cultured, passaged, differentiated, induced, etc., from cells isolated from skin tissue. “Derived from,” when used with respect to cells, refer to cells isolated from tissue and cells cultured, passaged, differentiated, induced, etc., from cells isolated from tissue.

By “pluripotency” it is meant the ability of cells to differentiate into all types of cells in an organism. By “pluripotent stem cells”, it is meant cells that can self-renew and differentiate to produce all types of cells in an organism. By “multipotency” it is meant the ability of cells to differentiate into some types of cells in an organism but not all, typically into cells of a particular tissue or cell lineage.

“Bind,” bound,” “binds to,” and “binding,” when used with respect to cell surface markers, refer to detectable binding of a molecule with a binding affinity and/or specificity for a cell surface marker. A cell having a cell surface marker, the binding of which to a molecule with a binding affinity and/or specificity for a cell surface marker is detectable, can be said to bind to the molecule. By “selectively bind” is meant that the molecule binds preferentially to the target of interest or binds with greater affinity to the target than to other molecules. For example, an antibody can bind to a molecule that includes an epitope for which it is specific and not to unrelated epitopes.

“Express,” “expression,” and “expressing,” when used with respect to gene products, indicate that the gene product of interest is expressed to a detectable level. “Significant expression” refers to expression of the gene product of interest to 10% above the minimum detectable expression. Cells with “high expression” or “high levels” of expression of a given expression product are the 10% of cells in a given sample or population of cells that exhibit the highest expression of the expression product. For example, skin cells with high expression of SSEA3 are the 10% of skin cells in a sample of skin cells that exhibit the highest expression of SSEA3. Such cells can be referred to as SSEA3^{HIGH}. Cells with “low expression” of a given expression product are the 10% of cells in a given sample or population of cells that exhibit the lowest expression of the expression product (which can be no expression). For example, skin cells with low expression of SSEA3 are the 10% of skin cells in a sample of skin cells that exhibit the lowest expression of SSEA3. Such cells can be referred to as SSEA3^{NEGATIVE}.

A cell “can differentiate into” a specified type of cell if, under conditions that induce differentiation of cells known to differentiate into the specified type of cell, the cell differentiates into the specified type of cell. A cell “does not differentiate into” a specified type of cell if, under conditions that induce differentiation of cells known to differentiate into the specified type of cell, the cell fails to differentiates into the specified type of cell. The ability to differentiate into a specified type of cell (or the lack of such ability) can be limited to one or several differentiation conditions. Thus, for example, a cell could be characterized as capable of differentiating into an osteoblast under the conditions used in the hMSC osteogenic differentiation BULLETKIT™ (Lonza, Cat. No. PT-3002), even though the cell might not differentiate into osteoblasts under different differentiation conditions. “Conditions that induce differentiation” of cells into a specified type of cell are conditions that cause cells known or established to differentiate to the specified cell type to differentiate into the specified cell type.

“Skin regeneration cell” refers to cells that mediate skin regeneration. “Mediate regeneration” and “participate in repair,” when used with respect to cells, refer to cells that cause or aid in regeneration and/or repair of tissue. For example, cells that mediate skin regeneration can be the source of new skin cells and/or can stimulate other cells to be the source of new skin cells.

The term “somatic cell” encompasses any cell in an organism that cannot give rise to all types of cells in an organism; that is, the cell is not pluripotent. For example, somatic cells are cells that have differentiated sufficiently that they will not naturally generate cells of all three germ layers of the body (that is, ectoderm, mesoderm and endoderm). Examples of somatic cells include those from ectodermal (for example, keratinocytes), mesodermal (for example, fibroblast), endodermal (for example, pancreatic cells), or neural crest lineages (for example, melanocytes). Somatic cells include, for example, dermal fibroblasts, keratinocytes, pancreatic beta cells, neurons, oligodendrocytes, astrocytes, hepatocytes, hepatic stem cells, cardiomyocytes, skeletal muscle cells, smooth muscle cells, hematopoietic cells, osteoclasts, osteoblasts, pericytes, vascular endothelial cells, Schwann cells, and the like. Somatic cells are cells that, in the absence of experimental manipulation, will not proliferate; or if they do, will only be able to give rise to more of their own kind (for example, terminally differentiated cells). Somatic cells can be cells that are differentiated to the point that they are capable of giving rise to cells of a specific lineage (for example, adult non-pluripotent multipotent stem cells, such as mesenchymal stem cells, neural stem cells, cardiac stem cells, hepatic stem cells, and the like). Somatic cells can have a phenotype reflective of their differentiated state (for example, markers, cell morphology, and/or functional characteristics that reflect the differentiated state of the cells).

As used herein, “subject” includes, but is not limited to, animals. The subject can be a vertebrate, more specifically a mammal (for example, a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, stabilize, or prevent one or more symptoms of a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes prophylactic or palliative treatment, that is, treatment designed for the relief of one or more symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization, or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

“Effective amount” of a cell, device, composition, or compound refers to a nontoxic but sufficient amount of the cell, device, composition, or compound to provide the desired result. The exact amount required will vary from subject to subject, depending on the species, age, and general condition of the subject, the severity of the disease that is being treated, the particular cell, device, composition, or compound used, its mode of administration, and the like. Thus, it is not possible to specify an exact “effective amount.” However, an appropriate effective amount can be determined by one of ordinary skill in the art using only routine experimentation.

“Pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable platform” refers to pharmaceutically acceptable materials, compositions or structures involved in holding, carrying, transporting, or delivering any subject cell or composition. Each platform must be “acceptable” in the sense of being compatible with the other ingredients of a subject composition and not injurious to the patient.

“Biocompatible” refers to one or more materials that are neither themselves toxic to the host (for example, an animal or human), nor degrade (if the material degrades) at a rate that produces monomeric or oligomeric subunits or other byproducts at toxic concentrations in the host.

“Biodegradable” means that the materials degrades or breaks down into its component subunits, or digestion, for example, by a biochemical process, of the material into smaller (for example, non-polymeric) subunits.

“Adult stem cells” are any type of stem cell that is not derived from an embryo or fetus. In their natural state, these stem cells generally have a limited capacity to generate new cell types (which can be referred to as “multipotency”) and are committed to a particular lineage. However, adult stem cells capable of generating all three cell types have been described (for example, U.S. Patent Application Publication No 20040107453 and PCT/US02/04652). Examples of adult stem cells are adipose-derived mesenchymal stem cells and multipotent hematopoietic stem cells. Multipotent hematopoietic stem cells form all of the cells of the blood, such as erythrocytes, macrophages, T and B cells. Cells such as these are referred to as “pluripotent hematopoietic stem cell” for its pluripotency within the hematopoietic lineage. A pluripotent adult stem cell is an adult stem cell having pluripotent capabilities (See for example, U.S. Patent Publication No: 2004/0107453).

The term “induced pluripotent stem cell” encompasses pluripotent stem cells, that, like embryonic stem (ES) cells, can be cultured over a long period of time while maintaining the ability to differentiate into all types of cells in an organism, but that, unlike ES cells (which are derived from the inner cell mass of blastocysts), are derived from somatic cells. Generally, pluripotent stem cells are cells that had a narrower, more defined potential and that, in the absence of experimental manipulation, could not give rise to all types of cells in the organism. iPS cells have a hESC-like morphology, growing as flat colonies with large nucleo-cytoplasmic ratios, defined borders and prominent nuclei. In addition, iPS cells can express one or more key pluripotency markers known to those of skill in the art, including but not limited to Alkaline Phosphatase, SSEA3, SSEA4, Sox2, Oct314, Nanog, TRA1S0, TRA181, TDGF 1, Dnmt3b, FoxD3, GDF3, Cyp26a1, TERT, and zfp42. In addition, the iPS cells can be capable of forming teratomas. In addition, iPS cells can be capable of forming or contributing to ectoderm, mesoderm, or endoderm tissues in a living organism.

By “having the potential to become iPS cells” it is meant that somatic cells can be induced to become, that is, can be reprogrammed to become, iPS cells. In other words, the somatic cell can be induced to redifferentiate so as to establish cells having the morphological characteristics, growth ability and pluripotency of pluripotent cells.

The term “efficiency of reprogramming” is used to refer to the ability of a primary cell culture to give rise to iPS cell colonies when contacted with reprogramming factors. By “enhanced efficiency of reprogramming” it is meant that the cells will demonstrate an enhanced ability to give rise to iPS cells when contacted with reprogramming factors relative to a control.

“Reprogramming factors” refers to one or more factors (that is, a cocktail) of biologically active factors that act on a cell to alter transcription, thereby reprogramming a cell to multipotency or to pluripotency. Reprogramming factors can be provided to cells individually or as a single composition (that is, as a premixed composition) of reprogramming factors. The factors can be provided at the same molar ratio or at different molar ratios. The factors can be provided once or multiple times in the course of culturing the cells. The reprogramming factor can be a transcription factors, including without limitation, Oct3/4; Sox2; Klf4; c-Myc; Nanog; and Lin-28.

It is to be understood that the disclosed cells, methods, and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### II. Cells

A. CD146 and CD271-Expressing Mesenchymal Stem Cells

Adult stem cell (ASC)-like subpopulations have been well characterized in the epidermis of mammalian skin (Fuchs (2009) Cell Stem Cell, 4: 499-502; Tumbar et al. (2004) Science, 303: 359-363; Blanpain et al. (2004) Cell, 118: 635-648). However, ASC-like subpopulations in the dermis of human skin are less well characterized. Previous research has demonstrated that the CD146 and CD271 cell surface biomarkers can be used to identify and isolate a subpopulation of multipotent stem cells from primary human adherent dermal (PHAD) cells (Zannettino et al. (2008) J Cell Physiol., 214: 413, Vaculik et al. (2012) J. Invest. Dermatol., 132: 563-574) although it was not known if these human skin multipotent stem cells play a direct role in dermal tissue regeneration. CD271 belongs to the low-affinity neurotrophin receptors and the tumor necrosis factor receptor superfamily. Initially the human CD271 (LNGFR) was described to be expressed on cells of the central and peripheral nervous system, and was suggested to be involved in the development, survival, and differentiation of cells (Thomson et al. (1988) Exp. Cell Res. 174: 533-539; Rogers et al. (2008) J. Biol. Regulators & Homeostatic Agents 22(1): 1-6).

A new form of mesenchymal stem cells (MSCs) has been discovered as a rare subpopulation of primary human skin cell cultures. This subpopulation is characterized by the cell surface biomarker CD 146 and CD271. These CD 146- and CD271-expressing cells can be cultured and then differentiated to various types of multipotent mesenchymal stem cells, such as human osteogenic dermal mesenchymal stem cells (HOD-MSCs). Primary human skill cell cultures are more convenient to obtain than other sources of human MSCs and so CD146-MSCs and CD271-MSCs can be a preferred alternative. CD146-MSCs and CD271-MSCs can be used in any of the ways that MSCs can be used, such as for storage, banking, diagnostics, research, tissue replacement, tissue repair, bone replacement, bone repair, such as spinal fusion and joint repair, replacement of degenerated tissue, etc. Thus, CD146-MSCs and CD271-MSCs have applications for treatment of, for example, destructive and degenerative injuries and diseases.

The CD146-MSCs and CD271-MSCs capable of differentiating into a variety of cell types, including for example, osteoblasts, chondrocytes, and adipocytes, exist as a rare population in skin dermis. CD146-MSCs and CD271-MSCs can be isolated from dermis using the marker CD146 and CD271 respectively, for example, as described in the Examples. CD146-MSCs and CD271-MSCs can be further isolated using one or markers such as CD10, CD13, CD26, CD29, CD34, CD44, CD54, CD71, CD73, CD90, CD105 (clone 266), CD106, CD166, ITGA11, STRO-1, and SSEA4.

CD146-MSCs and CD271-MSCs can be obtained by enriching CD146- and CD271-expressing cells from primary human skin cell cultures and growing the enriched CD146- and CD271-expressing cells to confluence. This method provides an efficient way to obtain CD146-MSCs and CD271-MSCs. Primary human skin cell cultures can be from any source and can be obtained using known and standard techniques. An example is described in the Examples below. Known culture techniques for cells, stem cells, and differentiation can be used with the CD146-MSCs and CD271-MSCs to achieve similar purposes as with their known uses with other MSCs.

CD146-MSCs and CD271-MSCs can be cultured and propagated using standard methods known to those of skill in the art.

CD146-MSCs and CD271-MSCs are multipotent stem cells that under certain conditions can differentiate into a variety of cell types, including: osteoblasts (bone cells), chondrocytes (cartilage cells), and adipocytes (fat cells)). Due to their multipotency in differentiation, mesenchymal stem cells are useful as xenograft, homograft and autograft materials for regenerative medicine of many tissues, such as bone, cartilage, tendon, muscle, fat, and periodontal tissue. These MSCs can be utilized in a variety of applications including, for example wound repair. It is preferred that the CD146-MSCs and CD271-MSCs be grown to confluence before inducing differentiation. It is preferred that optimized differentiation conditions be used when generating differentiated cells from CD146-MSCs and CD271-MSCs.

Human osteogenic dermal mesenchymal stem cells (HOD-MSCs) can be derived from CD146-MSCs and CD271-MSCs. HOD-MSCs can be derived from CD146-MSCs and CD271-MSCs using any suitable technique. For example, osteogenesis differentiation techniques can be used, such as the hMSC osteogenic BULLETKIT™ from Lonza. HOD-MSCs can be used in a variety of ways, such as for storage, banking, diagnostics, research, tissue replacement, tissue repair, bone replacement, bone repair, such as spinal fusion and joint repair, replacement of degenerated bone tissue, etc. Osteoblasts and osteocytes derived from CD146-MSCs, CD271-MSCs and HOD-MSCs can be used for treatment, repair, replacement, regeneration, etc. of bone tissue. Chondrocytes derived from CD146-MSCs, CD271-MSCs and HOD-MSCs can be used for treatment, repair, replacement, regeneration, etc. of cartilaginous tissue, such as ligaments and ear and nose scaffolding.

CD146-MSCs, CD271-MSCs can also be used to produce a variety of multipotent mesenchymal stem cells and a variety of differentiated cells. For example, CD146-MSCs and CD271-MSCs can be used in osteogenesis differentiation, adipogenesis differentiation, chondrogenesis differentiation, neurogenesis differentiation, cardiogenesis differentiation, beta cell differentiation, etc. Differentiation can occur in vitro, ex vivo, or in vivo.

B. SSEA3-Expressing Regeneration-Associated (SERA) Cells

Stage specific embryonic antigen 3 (SSEA3) is a cell surface glycosphingolipid that plays an important role in identifying cells with either cancer associated (Schrump et al. (1988) Proc. Natl. Acad. Sci. USA, 85: 4441-4445; Chang et al. (2008) Proc. Natl. Acad. Sci. USA, 105: p. 11667-11672; Ohyama et al. (1995) Cancer, 76: 1043-1050) or stem cell/undifferentiated cell characteristics (Shevinsky et al. (1982) Cell, 30: 697-705; Kannagi et al. (1983) Embo J., 2: 2355-2361; Byrne et al. (2009) PLoS ONE, 4: e7118; Kuroda et al. (2010) Proc. Natl. Acad. Sci. USA, 107: 8639-8643; Tonge et al. (2011) Stem Cell Res, 7: 145-53; Wakao et al. (2011) Proc. Natl. Acad. Sci. USA, 108: 9875-9880; Atlasi et al. (2008) Stem Cells, 26: 3068-3074), such as human embryonic carcinoma (EC) cells (Kannagi et al. (1983), supra), undifferentiated human embryo inner cell mass (ICM) cells (Henderson et al. (2002) Stem Cells, 20: 329-337), human embryonic stem (ES) cells (Thomson et al. (1998) Science, 282: 1145-1147) and human induced pluripotent stem (iPS) cells (Takahashi et al. (2007) Cell, 131: 861-872). The SSEA3 epitope was discovered by Solter and colleagues in 1982 as a biomarker expressed on oocytes, embryonic cells and cancer cells (Shevinsky et al. (1982), supra). In this initial report, Solter noted that SSEA3 was also expressed on erythrocytes (red blood cells) providing the first evidence that SSEA3 expression was not an exclusive cancer or stem/undifferentiated cell marker and can serve other functions in the body in a spatial, niche and tissue dependent manner.

Reports of SSEA3-expressing cells within human tissues such as kidney (Sekine et al. (1987) J. Biochem., 101: 563-568), brain (Dasgupta et al. (1995) J. Neurochem., 65: 2344-2349) and skin (Byrne et al. (2009), supra) raised the possibility that the cell surface expression of the SSEA3 epitope could facilitate the identification and isolation of ASC-like and/or undifferentiated cell subpopulations from adult human tissues. While SSEA3 has already been used to identify human skin-derived cells that are easier to reprogram to pluripotency (Byrne et al. (2009), supra; Wakao et al. (2011), supra; suggesting a less differentiated state), associated with multi-lineage differentiation potential (Kuroda et al. (2010) supra; suggesting a multipotent or possibly pluripotent state), and correlated to the occlusion of human blood vessels (Zulli, et al. (2008) Hum. Pathol., 39: 657-665; suggesting a possible regeneration-associated state for blood vessels), SSEA3 has not been investigated as a cell surface biomarker facilitating the identification and isolation of human skin cells directly associated with tissue regeneration following dermal injury.

It was determined that the SSEA3 biomarker can be used to facilitate the identification and isolation of regeneration-associated cells from adult human dermis. The Examples below present the first evidence directly correlating the expression of the SSEA3 glycosphingolipid on the surface of human skin cells to significantly increased proliferation in response to a dermal tissue injury/regenerative response.

A rare subpopulation of SSEA3 expressing cells was isolated that exists in the dermis of adult human skin. These SSEA3-expressing cells undergo a significant increase in cell number in response to injury, which led to the present discovery of a role for these cells in regeneration. These SSEA3-expressing regeneration-associated (SERA) cells were derived through primary cell culture, purified by fluorescence activated cell sorting (FACS) and characterized. The SERA cells demonstrated a global transcriptional state most similar to bone marrow and fat derived mesenchymal stem cells (MSCs) and the highest expressing SSEA3 expressing cells co-expressed CD105 (clone 35). SERA cells are particularly characterized by binding to clone 35 anti-CD105 antibodies (BD Biosciences). SERA cells do not significantly bind to clone 266 anti-CD105 antibodies (BD Biosciences). Recognition of the SERA cell-related CD105 biomarker by different forms of anti-CD105 antibodies indicates that the SERA cell-related form of CD105 differs from other forms of CD105. CD105 clone 35 binding is a preferred basis for identifying and isolating SERA cells.

While a rare population of MSCs was observed in primary human skin cell cultures that could differentiate into adipocytes, osteoblasts or chondrocytes (the CD271-MSCs described herein), SERA cells did not possess this differentiation capacity, leading to the discovery that there are at least two different rare subpopulations in adult human skin primary cultures. The identification, efficient purification and large-scale expansion of these rare subpopulations (SERA cells, CD146-MSCs and CD271-MSCs) from heterogeneous adult human skin primary cell cultures have applications for patient-specific cellular therapies.

Because SERA cells are associated with regeneration and wound repair, SERA cells can be used in, for example, the treatment of wounds, the preparation of synthetic graft materials, grafts, the augmentation of skin grafts, and the like.

The derivation of SERA cells, CD146-MSCs and/or CD271-MSCs from skin punch biopsies offer numerous advantages. Notably skin punch biopsies (and other skin biopsy methods), are quick, simple, and minimally invasive to perform. They are preferable for many patients to current MSC derivation protocols that require the surgical aspiration of adipose tissue or bone marrow.

Using the methods described herein, SERA cells, CD146-MSCs and/or CD271-MSCs, can be rapidly derived from primary cultures of skin samples, propagated and expanded ex vivo, and reintroduced into the subject for the treatment of various wounds and related conditions.

C. Cell Lines, Cell Cultures, and Cell Strains

CD146-MSCs, CD271-MSCs and SERA cells can be developed into cell lines, cell cultures, and cell strains. Similarly, cells differentiated from CD146-MSCs, CD271-MSCs and SERA cells can be developed into cell lines, cell cultures, and cell strains. CD146-MSCs, CD271-MSCs and SERA cells reprogrammed to induced pluripotent stem cells can be maintained as cell lines, cell cultures, and cell strains. Maintenance, storage, banking, culturing, proliferating, etc., of SERA cells, CD146-MSCs, CD271-MSCs, and/or cells derived from the SERA cells, CD146-MSCs and/or CD271-MSCs can be accomplished using standard techniques and media. Useful techniques and media are those used with mesenchymal stem cells for undifferentiated CD146-MSCs, CD271-MSCs and SERA cells. Techniques and media used with differentiated cells can be used with differentiated cells derived from CD146-MSCs, CD271-MSCs and SERA cells, with techniques and media for the particular type of differentiated cell being preferred.

Disclosed are cell lines, cell cultures, and cell strains that include a plurality of cells proliferated from the CD146-MSCs, CD271-MSCs or SERA cells. Also disclosed are cell lines, cell cultures, and cell strains that include a plurality of cells differentiated from the CD146-MSCs, CD271-MSCs or SERA cells. Also disclosed are cell lines and cell strains that include a plurality of induced pluripotent stem cells (iPSCs) derived ex vivo from the CD146-MSCs, CD271-MSCs or SERA cells. Also disclosed are cell lines and cell strains that include a plurality of cell differentiated ex vivo from iPSCs derived from the CD146-MSCs, CD271-MSCs or SERA cells.

D. Devices and Compositions Including Cells

The SERA cells, CD146-MSCs, CD271-MSCs, and/or cells derived from the SERA cells, CD146-MSCs, CD271-MSCs, or all three can be administered to a patient by way of a composition that includes the cells alone or on or in a carrier or support structure. In many cases, no carrier will be required. In these cases, the cells typically have been washed to remove cell culture media and will be suspended in a physiological buffer. The cells can be administered alone or in combination with other pharmaceutical or bioactive agents.

1. Therapeutic and or Prophylactic Compositions

Cell compositions can be therapeutic compositions. Therapeutic compositions can include the cells and a pharmaceutically acceptable platform. The therapeutic component can include a cellular component and a non-cellular component. The cellular component can be any of the disclosed cells. Therapeutic compositions that contain (a) SERA cells, CD146-MSCs, CD271-MSCs, and/or cells derived from the SERA cells, CD146-MSCs, CD271-MSCs, or both and (b) a pharmaceutically acceptable platform are provided. The cell compositions can be infused or injected systemically or locally as may be appropriate for the type of cell and the disease, condition or object. The cell composition containing, for example, SERA cells can be suspended in an appropriate medium for use as a cosmetic formulation.

The cell compositions can also include stabilizing agents, cell-affecting agents, therapeutic agents, or a combination. Cell-affecting agents are compounds, molecules, and compositions that affect cell growth, development, and/or proliferation. For example, cytokines can be included to stimulate cell and tissue alterations useful for the cells and/or therapeutic purpose. An agent can be both a cell-affecting agent and a therapeutic agent. Such agents can be referred to as pharmaceutically active agents. Useful pharmaceutically active agents can include, but are not limited to humoral factors to promote cell transplantation and engraftment. Useful active agents can also include, but are not limited to growth factors, anti-inflammatories, antibiotics, and antivirals. Growth factors include, but are not limited, to the TGF superfamily of growth factors, FGF, basic FGF, VEGF, insulin-like growth factor, EGF, PDGF, and nerve growth factor.

The non-cellular component of the therapeutic component of the composition can include biologically active agents in addition to those secreted by SERA cells, CD146-MSCs and/or CD271-MSCs and their derivatives. Illustrative biologically active agents include, but are not limited to proteins, peptides, sugars, glycoproteins, nucleic acids, small molecule therapeutics, prophylactic or diagnostic agents, and the like. Additional illustrative, biologically active agents include, but are not limited to angiogenic factors, inducers of differentiation, antibiotic and/or antimicrobials, and the like. Also contemplated are hormones, steroids, anti-inflammatories, analgesics, immunosuppressants, anticoagulants, muscle relaxants and antispasmodics, growth factors, colony stimulating factors, nutrients such as vitamins, small drug molecules, gene therapy agents, for example, plasmids, retrovirals, and combinations thereof, and other pharmacologically acceptable excipients, diluents, buffers, preservatives, and the like. Other applicable therapeutic agents include, but are not limited, to cytokines, morphogens, stem cells, umbilical cord stem cells, embryonic stem cells, adult stem cells, pluripotent cells derived from bone marrow, bone marrow stem cells, osteoblasts, osteoclasts, or a combination thereof. The cells can be autologous or heterologous. Cytokines include, but are not limited to, IL-1 through IL-13.

The cells can be provided in a matrix formulation or scaffold to facilitate cell survival, maintenance, and/or growth. For example, the cells can be provided in a fibrin scaffold as described, for example, in U.S. Patent Publication No: 2012/0039855. For example, a fibrin scaffold is provided that is capable of supporting or anchoring embedded MSC or other cells (for example, SERA cells) to an implantation site for a therapeutically effective period of time. In particular, the fibrin scaffold described therein facilitates blood vessel formation at a given implantation site of a subject. In some forms, the fibrin scaffold can be formed with a fibrinogen component containing fibrinogen having a final concentration of higher than 17.5 mg/ml scaffold. In particular, the fibrin scaffold can be capable of supporting a population of mesenchymal stem cells (MSC) and/or other derived cells seeded to a concentration of at least 1×106 cells/ml scaffold. In some forms, the scaffold can also have a biologically active component. For example, the scaffold can include a solution of proteins derived from blood plasma that can also have anti fibrinolytic agents such as tranexamic acid and/or stabilizers such as arginine, lysine, their pharmaceutically acceptable salts, or mixtures thereof. The solution can have additional factors such as, for example, factor VIII, fibronectin, von Willebrand factor (vWF), vitronectin, etc. Examples of this are described in U.S. Pat. No. 6,121,232 and WO 9833533. The solution can also have stabilizers such as tranexamic acid and arginine hydrochloride.

Implants fabricated from polymers and/or other scaffold materials can be used in a wide range of orthopedic and vascular applications, tissue engineering, guided tissue regeneration. There are tissue engineering applications for virtually every tissue, including liver, cartilage, kidney, lung, skin, heart, bladder, pancreas, bone, uroepithelial-smooth muscle structures (especially ureters and urethras), tracheal epithelium, tendon, breast, arteries, veins, heart valves, gastrointestinal tubes, fallopian tubes, bile ducts, esophagus, and bronchi.

Scaffolds are typically formed of polymeric, ceramic, and/or metals. Polymers may be in the form of fibers, sheets, woven or non-woven structures, hydrogels, or combinations thereof. A hydrogel is defined as a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include proteins such as fibrinogen, collagen, and hyaluronic acid, polysaccharides such as alginate, polyphosphazines, and polyacrylates, which are crosslinked ionically, or block copolymers such as PLURONICS® or TETRONICS®, polyethylene oxide-polypropylene glycol block copolymers which are crosslinked by temperature or pH, respectively. In general, these polymers are at least partially soluble in aqueous solutions, such as water, buffered salt solutions, or aqueous alcohol solutions, that have charged side groups, or a monovalent ionic salt thereof. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

Polymeric scaffolds may be formed of natural polymers such as fibrin or collagen or synthetic polymers, typically biodegradable polymers such as polyhydroxy acids like polylactic acid, polyglycolic acid and poly lactic-co-glycolic acid, polyhydroxyalkanoates such as poly(4-hydroxybutyrate), and polyacrylic acids. Other biocompatible, biodegradable materials include, but are not limited to, type 1 collagen, Poly-DL-lactide-caprolactone (PCL), laminin, and gelatin.

Any other suitable scaffold/matrix materials can be utilized to contain and apply the disclosed cells to a wound site. In some forms, such scaffold/matrix materials can have a natural or synthetic cell extracellular matrix material (ECM). Such extracellular matrix materials are well known to those of skill in the art (see, for example, Halstenberg et al. (2002) Biomacromolecules, 3: 710-723; Mann et al. (2001) Biomaterials, 22: 3045-3051, and the like). Illustrative synthetic ECM materials include, for example, hydrogel ECMs formed from biological materials (for example, hyaluronic and collagen hydrogels, see, for example, HYSTEM® hydrogels) or ECMs formed from synthetic hydrogel materials (for example, PEG-tetravinylsulfone, see, for example, Lutolf et al. (2003) Proc. Natl. Acad. Sci., USA, 100(9): 5413-5418, and the like).

Synthetic skins are contemplated that include a scaffold and a therapeutic component. The scaffold typically can be a matrix of fibers that forms interstices. The therapeutic component can include a cellular component and, optionally, a non-cellular component. The cellular component can include, for example, SERA cells, and/or cells derived from the SERA cells, CD146-MSCs, CD271-MSCs, or all three. In some forms, the fiber material can be a synthetic structural material such as a biocompatible polymer. Biodegradable polymers are also desirable. Such materials can be selected such that they are dissolved or resorbed by the body without the need for surgical removal procedures. Biocompatible, biodegradable materials useful in the grafts include, but are not limited to, polyglycolic acid (PGA), type 1 collagen, Poly-DL-lactide-caprolactone (PCL), laminin, gelatin, and the like. Methods of making artificial skins are described in U.S. Patent Publication Nos: 2012/0015022, 2011/0318414, 2011/0052693, 2009/0232878, 2008/0311613, 2008/0248571, 2008/0033550, 2004/0162615, 2004/0038859, 2003/0109927, 2002/0193875, 2002/0082692., which are incorporated herein by reference for the artificial skins and synthetic methods described therein.

2. Grafts and Implants

The cells can also be administered in devices including but not limited to rods, pins, screws, braces, plates, prosthesis or the like. The devices can be used for joining or fusing parts of one or more bones, joining tissue to bone, or joining tissue to tissue. The devices can also be used as a framework or scaffold for tissue growth. Such devices are useful, for example, for tissue replacement and regeneration. In addition to the cells, the devices and implants can be made of artificial materials, natural materials, or a combination. For example, in the devices can contain segments prepared from natural materials, synthetic materials (including polymers and ceramics), metals, metal alloys, or a combination thereof. In some forms, the device or implant can be made of titanium.

For bone and cartilage repair, regeneration, and/or replacement of bone, tendons, and cartilage, the device can include osteoblasts, osteocytes, or both. The devices can also include natural materials. As used herein, “natural material” can be any material derived from a natural source. For example, the natural material can be bone and cartilage, including bone and cartilage harvested from humans or animals. The bone used can also be heterologous, homologous and autologous (that is, xenograft, allograft, autograft) bone derived from, for example, fibula, tibia, radius, ulna, humerus, cranium, calcaneus, tarsus, carpus, vertebra, patella, ilium, etc. The bone can also be one or more bone products that have been partially or completely demineralized, prepared for transplantation (for example, via removal of immunogenic proteins), and/or processed by other techniques. Additionally, the implants can be prepared from products made from bone, such as chips, putties, and other similar bone products. Human source bone is preferred for human applications.

3. Tissue Engineering Constructs

For tissue engineering, the cells can be provided with or incorporated onto or into a support structure for construction of a new tissue. Support structures can be meshes, solid supports, tubes, porous structures, and/or a hydrogel. The support structures can be biodegradable or non-biodegradable, in whole or in part. The support can be formed of a natural or synthetic polymer, metal such as titanium, bone or hydroxyapatite, or a ceramic. Natural polymers include collagen, hyaluronic acid, polysaccharides, and glycosaminoglycans. Synthetic polymers include polyhydroxyacids such as polylactic acid, polyglycolic acid, and copolymers thereof, polyhydroxyalkanoates such as polyhydroxybutyrate, polyorthoesters, polyanhydrides, polyurethanes, polycarbonates, and polyesters.

a. Solid Supports

The support structure can be a loose woven or non-woven mesh, where the cells are seeded in and onto the mesh. The structure can include solid structural supports. The support can be a tube, for example, a neural tube for regrowth of neural axons. The support can be a stent or valve. The support can be a joint prosthetic such as a knee or hip, or part thereof, that has a porous interface allowing ingrowth of cells and/or seeding of cells into the porous structure.

The support structure can be a permeable structure having pore-like cavities or interstices that shape and support the hydrogel-cell mixture. For example, the support structure can be a porous polymer mesh, a natural or synthetic sponge, or a support structure formed of metal or a material such as bone or hydroxyapatite. The porosity of the support structure should be such that nutrients can diffuse into the structure, thereby effectively reaching the cells inside, and waste products produced by the cells can diffuse out of the structure.

The support structure can be shaped to conform to the space in which new tissue is desired. For example, the support structure can be shaped to conform to the shape of an area of the skin that has been burned or the portion of cartilage or bone that has been lost. Depending on the material from which it is made, the support structure can be shaped by cutting, molding, casting, or any other method that produces a desired shape. The support can be shaped either before or after the support structure is seeded with cells or is filled with a hydrogel-cell mixture, as described below.

Additional factors, such as growth factors, other factors that induce differentiation or dedifferentiation, secretion products, immunomodulators, anti-inflammatory agents, regression factors, biologically active compounds that promote innervation or enhance the lymphatic network, and drugs, can be incorporated into the polymer support structure.

An example of a suitable polymer is polyglactin, which is a 90:10 copolymer of glycolide and lactide, and is manufactured as VICRYL™ braided absorbable suture (Ethicon Co., Somerville, N.J.). Polymer fibers (such as VICRYL™), can be woven or compressed into a felt-like polymer sheet, which can then be cut into any desired shape. Alternatively, the polymer fibers can be compressed together in a mold that casts them into the shape desired for the support structure. In some cases, additional polymer can be added to the polymer fibers as they are molded to revise or impart additional structure to the fiber mesh. For example, a polylactic acid solution can be added to this sheet of polyglycolic fiber mesh, and the combination can be molded together to form a porous support structure. The polylactic acid binds the crosslinks of the polyglycolic acid fibers, thereby coating these individual fibers and fixing the shape of the molded fibers. The polylactic acid also fills in the spaces between the fibers. Thus, porosity can be varied according to the amount of polylactic acid introduced into the support. The pressure required to mold the fiber mesh into a desirable shape can be quite moderate. All that is required is that the fibers are held in place long enough for the binding and coating action of polylactic acid to take effect.

Alternatively, or in addition, the support structure can include other types of polymer fibers or polymer structures produced by techniques known in the art. For example, thin polymer films can be obtained by evaporating solvent from a polymer solution. These films can be cast into a desired shaped if the polymer solution is evaporated from a mold having the relief pattern of the desired shape. Polymer gels can also be molded into thin, permeable polymer structures using compression molding techniques known in the art.

Many other types of support structures are also possible. For example, the support structure can be formed from sponges, foams, corals, or biocompatible inorganic structures having internal pores, or mesh sheets of interwoven polymer fibers. These support structures can be prepared using known methods.

b. Hydrogels

In certain embodiments the cells can be mixed with a hydrogel to form a cell-hydrogel mixture. This cell-hydrogel mixture can be applied directly to a tissue that has been damaged. For example, as described in U.S. Pat. No. 5,944,754, a hydrogel-cell mixture can simply be brushed, dripped, or sprayed onto a desired surface or poured or otherwise made to fill a desired cavity or device. The hydrogel provides a thin matrix or scaffold within which the cells adhere and grow. These methods of administration can be especially well suited when the tissue associated with a patient's disorder has an irregular shape or when the cells are applied at a distant site (for example, when the cells are placed beneath the renal capsule to treat diabetes).

Alternatively, the hydrogel-cell mixture can be introduced into a permeable, biocompatible support structure so that the mixture essentially fills the support structure and, as it solidifies, assumes the support structure's shape. Thus, the support structure can guide the development and shape of the tissue that matures from the implanted cells, or their progeny, that are placed within it. A hydrogel-based method for generating new tissue using isolated cells is described for example in U.S. Pat. No. 6,171,610, the contents of which are incorporated herein by reference.

The support structure can be provided to a patient either before or after being filled with the hydrogel-cell mixture. For example, the support structure can be placed within a tissue (for example, a damaged area of the skin, the liver, or the skeletal system) and subsequently filled with the hydrogel-cell composition using a syringe, catheter, or other suitable device. When desirable, the shape of the support structure can be made to conform to the shape of the damaged tissue. In the following subsections, suitable support structures, hydrogels, and delivery methods are described (cells suitable for use are described above).

The hydrogels should be biocompatible, biodegradable, capable of sustaining living cells, and, preferably, capable of solidifying rapidly in vivo (for example, in about five minutes after being delivered to the support structure). Large numbers of cells can be distributed evenly within a hydrogel; a hydrogel can support approximately 5×10⁶ cells/ml. Hydrogels also allow diffusion so that nutrients reach the cells and waste products can be carried away. A variety of different hydrogels can be used with the disclosed cells and compositions. These include, but are not limited to: (1) temperature dependent hydrogels that solidify or set at body temperature (e.g., PLURONICS™); (2) hydrogels cross-linked by ions (e.g., sodium alginate); (3) hydrogels set by exposure to either visible or ultraviolet light, (e.g., polyethylene glycol polylactic acid copolymers with acrylate end groups); and (4) hydrogels that are set or solidified upon a change in pH (e.g., TETRONICS™). Materials that can be used to form these different hydrogels include, but are not limited to, polysaccharides such as alginate, polyphosphazenes, and polyacrylates, which are cross-linked ionically, block copolymers such as PLURONICS™ (also known as POLOXAMERS™), which are poly(oxyethylene)-poly(oxypropylene) block polymers solidified by changes in temperature, TETRONICS™ (also known as POLOXAMINES™), which are poly(oxyethylene)-poly(oxypropylene) block polymers of ethylene diamine solidified by changes in pH.

For purposes of preventing the passage of antibodies into the hydrogel, but allowing the entry of nutrients, a useful polymer size in the hydrogel is in the range of between 10 and 18.5 kDa. Smaller polymers result in gels of higher density with smaller pores.

Ionic Hydrogels

In general, polymers that form ionic hydrogels are at least partially soluble in aqueous solutions (e.g., water, aqueous alcohol solutions that have charged side groups, or monovalent ionic salts thereof). There are many examples of polymers with acidic side groups that can be reacted with cations (e.g., poly(phosphazenes), poly(acrylic acids), and poly(methacrylic acids)). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

Polyphosphazenes are polymers with backbones consisting of nitrogen and phosphorous atoms separated by alternating single and double bonds. Each phosphorous atom is covalently bonded to two side chains. Useful polyphosphazenes can have a majority of side chains that are acidic and capable of forming salt bridges with di- or trivalent cations. Examples of acidic side chains are carboxylic acid groups and sulfonic acid groups. Bioerodible polyphosphazenes can have at least two different types of side chains: acidic side chains capable of forming salt bridges with multivalent cations, and side chains that hydrolyze in vivo (e.g., imidazole groups, amino acid esters, glycerol, and glucosyl). Bioerodible or biodegradable polymers (i.e., polymers that dissolve or degrade within a period that is acceptable in the desired application (usually in vivo therapy)), will degrade in less than about five years and most preferably in less than about one year, once exposed to a physiological solution of pH 6-8 having a temperature of between about 25° C. and 38° C. Hydrolysis of the side chain results in erosion of the polymer. Examples of hydrolyzing side chains are unsubstituted and substituted imidizoles and amino acid esters in which the side chain is bonded to the phosphorous atom through an amino linkage.

Methods for synthesis and the analysis of various types of polyphosphazenes are described in U.S. Pat. Nos. 4,440,921, 4,495,174, and 4,880,622. Methods for the synthesis of the other polymers described above are known to those of skill in the art. See, for example Concise Encyclopedia of Polymer Science and Engineering, J. I. Kroschwitz, Ed., John Wiley and Sons, New York, N.Y., 1990. Many polymers, such as poly(acrylic acid), alginates, and PLURONICS™ are commercially available.

Water soluble polymers with charged side groups can be cross-linked by reacting the polymer with an aqueous solution containing multivalent ions of the opposite charge, either multivalent cations if the polymer has acidic side groups, or multivalent anions if the polymer has basic side groups. Cations useful for cross-linking the polymers with acidic side groups to form a hydrogel include divalent and trivalent cations such as copper, calcium, aluminum, magnesium, and strontium. Aqueous solutions of the salts of these cations can be added to the polymers to form soft, highly swollen hydrogels.

Anions for cross-linking the polymers to form a hydrogel include divalent and trivalent anions such as low molecular weight dicarboxylate ions, terepthalate ions, sulfate ions, and carbonate ions. Aqueous solutions of the salts of these anions can be added to the polymers to form soft, highly swollen hydrogels, as described with respect to cations.

Ionic polysaccharides, such as alginates or chitosan, can also be used to suspend living cells, including the cells described herein and their progeny. These hydrogels can be produced by cross-linking the anionic salt of alginic acid, a carbohydrate polymer isolated from seaweed, with ions, such as calcium cations. The strength of the hydrogel generally increases with either increasing concentrations of calcium ions or alginate. U.S. Pat. No. 4,352,883 describes the ionic cross-linking of alginate with divalent cations, in water, at room temperature, to form a hydrogel matrix.

The cells can be mixed with an alginate solution, for example, which can be delivered to an already implanted support structure, and which can then solidify in a short time due to the presence of physiological concentrations of calcium ions in vivo. Alternatively, the solution can be delivered to the support structure prior to implantation and solidified in an external solution containing calcium ions.

Temperature-Dependent hydrogels

Temperature-dependent, or thermosensitive, hydrogels can also be used with the disclosed cells. These hydrogels have so-called “reverse gelation” properties, that is, they are liquids at or below room temperature, and gel when warmed to higher temperatures (e.g., body temperature). Thus, these hydrogels can be easily applied at or below room temperature as a liquid and automatically form a semi-solid gel when warmed to body temperature. As a result, these gels are especially useful when the support structure is first implanted into a patient, and then filled with the hydrogel-cell composition. Examples of such temperature-dependent hydrogels are PLURONICS™ (BASF-Wyandotte), such as polyoxyethylene-polyoxypropylene F-108, F-68, and F-127, poly(N-isopropylacrylamide), and N-isopropylacrylamide copolymers.

These copolymers can be manipulated by standard techniques to affect their physical properties such as porosity, rate of degradation, transition temperature, and degree of rigidity. For example, the addition of low molecular weight saccharides in the presence and absence of salts affects the lower critical solution temperature (LCST) of typical thermosensitive polymers. In addition, when these gels are prepared at concentrations ranging between 5 and 25% (W/V) by dispersion at 4° C., the viscosity and the gel-sol transition temperature are affected, the gel-sol transition temperature being inversely related to the concentration. These gels have diffusion characteristics capable of allowing cells to survive and be nourished. U.S. Pat. No. 4,188,373 describes using PLURONIC™ polyols in aqueous compositions to provide thermal gelling aqueous systems. U.S. Pat. Nos. 4,474,751 and 4,478,822 describe drug delivery systems that utilize thermosetting polyoxyalkylene gels. With these systems, both the gel transition temperature and/or the rigidity of the gel can be modified by adjustment of the pH and/or the ionic strength, as well as by the concentration of the polymer.

pH-Dependent hydrogels

Other hydrogels suitable for use with the disclosed cells are pH-dependent. These hydrogels are liquids at, below, or above specific pH values, and gel when exposed to specific pHs, for example, 7.35 to 7.45, the normal pH range of extracellular fluids within the human body. Thus, these hydrogels can be easily delivered to an implanted support structure as a liquid and automatically form a semi-solid gel when exposed to body pH. Examples of such pH-dependent hydrogels are TETRONICS™ (BASF-Wyandotte) polyoxyethylene-polyoxypropylene polymers of ethylene diamine, poly(diethyl aminoethyl methacrylate-g-ethylene glycol), and poly(2-hydroxymethyl methacrylate). These copolymers can be manipulated by standard techniques to affect their physical properties.

Light Solidified Hydrogels

Other hydrogels that can be used with the disclosed cells are solidified by either visible or ultraviolet light. These hydrogels are made of macromers including a water soluble region, a biodegradable region, and at least two polymerizable regions as described for example in U.S. Pat. No. 5,410,016). The hydrogel can begin with a biodegradable, polymerizable macromer including a core, an extension on each end of the core, and an end cap on each extension. The core can be a hydrophilic polymer, the extensions can be biodegradable polymers, and the end caps can be oligomers capable of cross-linking the macromers upon exposure to visible or ultraviolet light, for example, long wavelength ultraviolet light. Examples of such light solidified hydrogels include polyethylene oxide block copolymers, polyethylene glycol polylactic acid copolymers with acrylate end groups, and 10K polyethylene glycol-glycolide copolymer capped by an acrylate at both ends. As with the PLURONIC™ hydrogels, the copolymers of these hydrogels can be manipulated by standard techniques to modify their physical properties such as rate of degradation, differences in crystallinity, and degree of rigidity.

Preparation of Hydrogel-Cell Mixtures

Once a hydrogel of choice is prepared, the cells described herein are suspended in the hydrogel solution. The concentration of the cells suspended in the hydrogel solution can mimic that of the tissue to be generated. For example, the concentration of cells can range between 10 and 100 million cells/ml (e.g., between 20 and 50 million cells/ml or between 50 and 80 million cells/ml). The optimal concentration of cells to be delivered into the support structure can be determined on a case by case basis, and can vary depending on cell type and the region of the patient's body into which the support structure is implanted or onto which it is applied.

Administering Hydrogel-Cell Mixtures

The liquid hydrogel-cell mixture can be delivered to a shaped support structure, either before or after the support structure is implanted in or applied to a patient. The specific method of delivery will depend on whether the support structure is sufficiently “sponge-like” for the given viscosity of the hydrogel-cell composition, that is, whether the support structure easily retains the liquid hydrogel-cell mixture before it solidifies. Sponge-like support structures can be immersed within, and saturated with, the liquid hydrogel-cell mixture, and subsequently removed from the mixture. The hydrogel is then allowed to solidify within the support structure. The hydrogel-cell-containing support structure is then implanted in or otherwise administered to the patient. The support structure can also be applied to the patient before the hydrogel completely solidifies. Alternatively, a sponge-like support structure can be injected with the liquid hydrogel-cell mixture, either before or after the support structure is implanted in or otherwise administered to the patient. The hydrogel-cell mixture is then allowed to solidify.

Support structures that do not easily retain the liquid composition require somewhat different methods. In those cases, for example, the support structure is immersed within and saturated with the liquid hydrogel-cell mixture, which is then allowed to partially solidify. Once the cell-containing hydrogel has solidified to the point where the support structure can retain the hydrogel, the support structure is removed from the partially solidified hydrogel, and, if necessary, partially solidified hydrogel that remains attached to the outside of the support structure is removed (e.g., scraped off the structure).

Alternatively, the liquid hydrogel-cell mixture can be delivered into a mold containing the support structure. For example, the liquid hydrogel-cell mixture can be injected into an otherwise fluid-tight mold that contains the support structure and matches its outer shape and size. The hydrogel is then solidified within the mold, for example, by heating, cooling, light-exposure, or pH adjustment, after which, the hydrogel-cell-containing support structure can be removed from the mold in a form that is ready for administration to a patient.

The support structure can also be implanted in or otherwise administered to the patient (e.g., placed over the site of a burn or other wound, placed beneath the renal capsule, or within a region of the body damaged by ischemia), and the liquid hydrogel-cell mixture can then be delivered to the support structure. The hydrogel-cell mixture can be delivered to the support using any simple device, such as a syringe or catheter, or merely by pouring or brushing a liquid gel onto a support structure (e.g., a sheet-like structure).

To apply or implant the support structure, the implantation site within the patient can be prepared (e.g., in the event the support structure is applied to the skin, the area can be prepared by debridement), and the support structure can be implanted or otherwise applied directly at that site. If necessary, during implantation, the site can be cleared of bodily fluids such as blood (e.g., with a burst of air or suction).

Numerous materials, devices, and techniques have been developed for tissue regeneration and tissue engineering. These materials, devices, and techniques can be used with, and adapted for, the disclosed cells. Examples of tissue regeneration and tissue engineering techniques include those described in U.S. Patent Application Nos. 20120122767, 20120122222, 20120122219, 20120121793, 20120121531, 20120116568, 20120114763, 20120114755, 20120109613, 20120108516, 20120107403, 20120100108, 20120093914, 20120093879, 20120093801, 20120078379, 20120078297, 20120077272, 20120076786, 20120064050, 20120058093, 20120053689, 20120046601, 20120045770, 20120045487, 20120040461, 20120034191, 20120027807, 20120016491, 20120015439, 20120003271, 20120003193, 20110318414, 20110305760, 20110301525, 20110293712, 20110293685, 20110288656, 20110287071, 20110280914, 20110274742, 20110270221, 20110262518, 20110257726, 20110256628, 20110256204, 20110256117, 20110250688, 20110250585, 20110244572, 20110224800, 20110189285, 20120065358, 20110256205, 20110008277, 20100234244, 20080193536, 20080182240, 20080145338, 20060198827, 20040222102, and 20020072798; Sachlos and Czernuszka (2003) Eur. Cells Materials 5: 29-40; Ma and Elisseeff, “Scaffolding in Tissue Engineering” (CRC Press, 2005); and Chan and Leong (2008) Eur. Spine J. 17(Supp. 4): 467-479.

The foregoing scaffold/matrix materials are intended to be illustrative and not limiting. Using the teaching provided herein, numerous other scaffold materials suitable of application of SERS and/or MSCs described herein to a wound site will be available to one of skill in the art.

4. Kits

The materials described herein as well as other materials can be packaged together in any suitable combination as a kit useful for performing, or aiding in the performance of, the disclosed method. It is useful if the kit components in a given kit are designed and adapted for use together in the disclosed method. For example disclosed are kits for isolating SERA cells from skin cells, the kit including antibodies specific for detection and sorting of cells expressing SSEA3 and CD105 (clone 35). As another example, disclosed are kits for isolating CD146-MSCs and CD271-MSCs from skin cells, the kit including antibodies specific for detection and sorting of cells expressing CD146 and CD271. The kits also can contain media for proliferating, storing, differentiating, and/or inducing the cells. The kits can also contain materials for collection of cells.

Also disclosed are kits for preparing and/or packaging cells for delivery to a user, such as a surgeon or doctor. Such kits can be used, for example, to return populations of SERA cells, CD146-MSCs, CD271-MSCs, and/or cells derived from SERA cells, CD146 and/or CD271-MSCs.

Also disclosed are kits containing the cells disclosed herein for therapeutic/cosmetic use. For cosmetic applications, for example, the kit can contain dosage units of the cells described herein, preferably, SERA cells. The total cell count can range from 3.4×10⁸ to 1×10⁹ cells. For example, cryovials can be used to prepare the final dosage unit of the cells, formulated to the desired cell concentration and cryopreserved. Preferably, the kit contains appropriate media for reconstituting the cells prior to administration, for example, an injection diluents such as such as bacteriostatic water, sterile water, sodium chloride, or phosphate buffered saline. Alternatively, DMEM may be used as the diluent.

### III. Methods of Differentiation

A. Differentiated Cells

There are numerous methods of differentiating the CD146, CD271-MSCs, iPSCs, and reprogrammed SERA cells into a more specialized cell type. Methods of differentiating CD146, CD271-MSCs, iPSCs, and reprogrammed SERA cells can be similar to those used to differentiate stem cells, particularly ES cells, MSCs, MAPCs, MIAMI, hematopoietic stem cells (HSCs). In some cases, the differentiation occurs ex vivo; in some cases the differentiation occurs in vivo.

Any known method of generating neural stem cells from stem cells can be used to generate neural stem cells from the disclosed cells (see, for example, Reubinoff et al. (2001) Nat. Biotechnol., 19(12): 1134-40). For example, neural stem cells can be generated by culturing the disclosed cells as floating aggregates in the presence of noggin, or other bone morphogenetic protein antagonist, see for example, Itsykson et al. (2005), Mol. Cell Neurosci., 30(1): 24-36. As another example, neural stem cells can be generated by culturing the disclosed cells in suspension to form aggregates in the presence of growth factors, for example, FGF-2, Zhang et al. (2001) Nat. Biotech., (19): 1129-1133. In some cases, the aggregates can be cultured in serum-free medium containing FGF-2. The cells can be co-cultured with a mouse stromal cell line, for example, PAG in the presence of serum-free medium containing FGF-2. The cells can be directly transferred to serum-free medium containing FGF-2 to directly induce differentiation.

Neural stems derived from the disclosed cells can be differentiated into neurons, oligodendrocytes, or astrocytes. Often, the conditions used to generate neural stem cells can also be used to generate neurons, oligodendrocytes, or astrocytes.

Dopaminergic neurons play a central role in Parkinson's disease and other neurodegenerative diseases and are thus of particular interest. In order to promote differentiation into dopaminergic neurons, the disclosed cells can be co-cultured with a PA6 mouse stromal cell line under serum-free conditions (see, for example, Kawasaki et al. (2000) Neuron, 28(1): 3140. Other methods have also been described (see, for example, Pomp et al. (2005) Stem Cells 23(7): 923-30; U.S. Pat. No. 6,395,546, for example, Lee et al. (2000) Nature Biotechnol, 18: 675-679).

Oligodendrocytes can also be generated from the disclosed cells. Differentiation of the disclosed cells into oligodendrocytes can be accomplished by known methods for differentiating stem cells or neural stem cells into oligodendrocytes. For example, oligodendrocytes can be generated by co-culturing the disclosed cells (preferably neural stem cells derived from CD146-MSCs, CD271-MSCs or iPSCs derived from SERA cells) with stromal cells, for example, Hermann et al. (2004) J Cell Sci. 117(Pt 19): 4411-4422. Oligodendrocytes can be generated by culturing the disclosed cells (preferably neural stem cells derived from CD146-MSCs, CD271-MSCs or iPSCs derived from SERA cells) in the presence of a fusion protein, in which the Interleukin (IL)-6 receptor, or derivative, is linked to the IL-6 cytokine, or derivative thereof. Oligodendrocytes can also be generated from the disclosed cells by other methods known in the art see, for example, Kang et al. (2007) Stem Cells 25: 419-424.

Astrocytes can also be produced from the disclosed cells. Astrocytes can be generated by culturing disclosed cells (preferably neural stem cells derived from CD 146-MSCs, CD271-MSCs or iPSCs derived from SERA cells) in the presence of neurogenic medium with bFGF and EGF (see for example, Brustle et al. (1999) Science, 285: 754-756).

The disclosed cells can be differentiated into pancreatic beta cells by methods known in the art, for example, Lumelsky et al. (2001) Science, 292: 1389-1394; Assady et al. (2001) Diabetes, 50: 1691-1697; D'Amour et al. (2006) Nat. Biotechnol., 24: 1392-1401; D'Amour et al. (2005) Nat. Biotechnol. 23: 1534-1541. The method can involve culturing the disclosed cells in serum-free medium supplemented with Activin A, followed by culturing in the presence of serum-free medium supplemented with all-trans retinoic acid, followed by culturing in the presence of serum-free medium supplemented with bFGF and nicotinamide, for example, Jiang et al. (2007) Cell Res., 4: 333-444. The methods can involve culturing the disclosed cells in the presence of serum-tree medium, activin A, and Wnt protein from about 0.5 to about 6 days, for example, about 0.5, 1, 2, 3, 4, 5, 6, days; followed by culturing in the presence of from about 0.1% to about 2%, for example, 0.2%, FBS and activin A from about 1 to about 4 days, for example, about 1, 2, 3, or 4 days; followed by culturing in the presence of 2% FBS, FGF-10, and KAAD-cyclopamine (keto-N-aminoethylaminocaproyl dihydro cinnamoylcyclopamine) and retinoic acid from about 1 to about 5 days, for example, 1, 2, 3, 4, or 5 days; followed by culturing with 1% B27, gamma secretase inhibitor and extendin-4 from about 1 to about 4 days, for example, 1, 2, 3, or 4 days; and finally culturing in the presence of 1% B27, extendin-4, IGF-1, and HGF for from about 1 to about 4 days, for example, 1, 2, 3, or 4 days.

Hepatic cells or hepatic stem cells can be differentiated from the disclosed cells. For example, culturing the disclosed cells in the presence of sodium butyrate can generate hepatocytes (see for example, Rambhatla et al. (2003) Cell Transplant, 12: 1-11). In another example, hepatocytes can be produced by culturing the disclosed cells in serum-free medium in the presence of Activin A, followed by culturing the cells in fibroblast growth factor-4 and bone morphogenetic protein-2 (for example, Cal et al. (2007) Hepatology, 45(5): 1229-39). The cells can be differentiated into hepatic cells or hepatic stem cells by culturing the disclosed cells in the presence of Activin A from about 2 to about 6 days, for example, about 2, about 3, about 4, about 5, or about 6 days, and then culturing the disclosed cells in the presence of hepatocyte growth factor (HGF) for from about 5 days to about 10 days, for example, about 5, about 6, about 7, about 8, about 9, or about 10 days.

The cells can also be differentiated into cardiac muscle cells Inhibition of bone morphogenetic protein (BMP) signaling can result in the generation of cardiac muscle cells (or cardiomyocytes) (see, for example, Yuasa et al. (2005) Nat. Biotechnol., 23(5): 607-611). The cells can be cultured in the presence of noggin for from about two to about six days, for example, about 2, about 3, about 4, about 5, or about 6 days, prior to allowing formation of an embryoid body, and culturing the embryoid body for from about 1 week to about 4 weeks, for example, about 1, about 2, about 3, or about 4 weeks.

Cardiomyocytes can be generated by culturing the disclosed cells in the presence of leukemia inhibitory factor (LIF), or by subjecting them to other methods known in the art to generate cardiomyocytes from ES cells (for example, Bader et al. (2000) Circ. Res., 86: 787-794, Kehat et al. (2001) J. Clin. Invest., 108: 407-414; Mummery et al. (2003) Circulation, 107: 2733-2740).

Examples of methods to generate other cell-types from the disclosed cells include: (1) culturing the disclosed cells in the presence of retinoic acid, leukemia inhibitory factor (LIE), thyroid hormone (T3), and insulin in order to generate adipocytes (for example, Dani et al. (1997) J. Cell Sci., 110: 1279-1285); (2) culturing the disclosed cells in the presence of BMP-2 or BMP4 to generate chondrocytes (for example, Kramer et al. (2000) Mech. Dev., 92: 193-205); (3) culturing the disclosed cells under conditions to generate smooth muscle (for example, Yamashita et al. (2000) Nature, 408: 92-96); (4) culturing the disclosed cells in the presence of beta-1 integrin to generate keratinocytes (for example, Bagutti et al. (1996) Dev. Biol., 179: 184-196); (5) culturing the disclosed cells in the presence of Interleukin-3 (IL-3) and macrophage colony stimulating factor to generate macrophages (for example, Lieschke and Dunn (1995) Exp. Hemat., 23: 328-334); (6) culturing the disclosed cells in the presence of IL-3 and stem cell factor to generate mast cells (for example, Tsai et al. (2000) Proc. Natl. Acad. Sci. USA, 97: 9186-9190); (7) culturing the disclosed cells in the presence of dexamethasone and stromal cell layer, steel factor to generate melanocytes (for example, Yamane et al. (1999) Dev. Dyn., 216: 450-458); (8) co-culturing the disclosed cells with fetal mouse osteoblasts in the presence of dexamethasone, retinoic acid, ascorbic acid, beta-glycerophosphate to generate osteoblasts (for example, Buttery et al. (2001) Tissue Eng., 7: 89-99); (9) culturing the disclosed cells in the presence of osteogenic factors to generate osteoblasts (for example, Sottile et al. (2003) Cloning Stem Cells, 5: 149-155); (10) overexpressing insulin-like growth factor-2 in the disclosed cells and culturing the cells in the presence of dimethyl sulfoxide to generate skeletal muscle cells (for example, Prelle et al. (2000) Biochem. Biophys. Res. Commun., 277: 631-638); (11) subjecting the disclosed cells to conditions for generating white blood cells; or (12) culturing the disclosed cells in the presence of BMP4 and one or more: SCF, FLT3, IL-3, IL-6, and GCSF to generate hematopoietic progenitor cells (for example, Chadwick et al. (2003) Blood, 102: 906-915).

B. Induced Pluripotent Stem Cells (iPSCs)

The disclosed cells (SERA cells, CD146-MSCs, CD271-MSCs, and/or cells derived from the SERA cells, CD146-MSCs and/or CD271-MSCs) can be reprogrammed into more immature precursor cells including, but not limited to, induced pluripotent stem (iPS) cells. The resultant iPS cells generated can be normal (apart from the genes introduced into such cells in order to induce the dedifferentiation process). For example, induced pluripotent stem cells can be made by ectopically expressing a SOX2 nucleic acid and an OCT4 nucleic acid in the cells, and then culturing the cells under culture conditions and for a time sufficient to generate induced pluripotent stem cells derived from the cells (see, for example, U.S. Patent Application Publication No. US 2011/0151447). General methods of producing iPS cells are described below. Other methods of producing iPS cells are described in, for example, WO 2011/022507 and U.S. Patent Application Publication No. US 2011/0104805.

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for isolating SERA cells from skin cells, the kit including antibodies specific for detection and sorting of cells expressing SSEA3 and CD105 (clone 35). As another example, disclosed are kits for isolating CD146-MSCs and CD271-MSCs from skin cells, the kit including antibodies specific for detection and sorting of cells expressing CD146 and CD271. The kits also can include media for proliferating, storing, differentiating, and/or inducing the cells.

Disclosed are mixtures formed by performing or preparing to perform the disclosed methods. For example, disclosed are mixtures including primary human skin cell cultures and antibodies specific for detection and sorting of cells expressing SSEA3 and CD105 (clone 35). As another example, disclosed are mixtures including primary human skin cell cultures and antibodies specific for detection and sorting of cells expressing CD146 and CD271.

Whenever the methods involve mixing or bringing into contact compositions or components or reagents, performing the methods create a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures including any disclosed cell, reagent, composition, or component, for example, disclosed herein.

### IV. Methods of Isolating CD146-MSCs, CD271-MSCs and SERA Cells

CD146-MSCs and CD271-MSCs can be isolated by culturing cells from a tissue sample including dermis of human skin (or other mammalian skin), and isolating a subpopulation of the cultured cells that express CD146 and CD271 respectively. SERA cells can be isolated by culturing cells from a tissue sample including dermis of human skin (or other mammalian skin), and isolating a subpopulation of the cultured cells that express high levels of SSEA3 and that are bound by anti-human CD 105 antibody clone 35. The subpopulations can be cultured ex vivo.

CD146-MSCs and CD271-MSCs can be produced, for example, by obtaining a tissue sample from dermis of human skin (or other mammalian skin); culturing cells from the sample; and isolating a subpopulation of the cells that expresses CD 146 and CD271 respectively. SERA cells can be produced, for example, by obtaining a tissue sample from dermis of human skin (or other mammalian skin); culturing cells from the sample; and isolating a subpopulation of the cells that expresses high levels of SSEA3 and that are bound by anti-human CD105 antibody clone 35. CD146-MSCs, CD271-MSCs and SERA cells can be isolated from an in vitro culture of primary human skin cells. CD146-MSCs, CD271-MSCs and SERA cells can be cultured and stored ex vivo.

SERA cells are readily isolated from a primary culture of dermal cells (for example, dermal human fibroblast (HUF1) cells). As illustrated in the Examples, human primary skin-derived cells (for example, HUF1) can readily be obtained from a skin biopsy (for example, an adult skin punch biopsy). The cells are cultured using standard culture conditions well known to those of skill in the art ((for example, using Dulbecco's Modified Eagle Medium Nutrient Mixture F-12 (DMEMIF12) supplemented with 10% fetal bovine serum (FBS, Invitrogen) and 100 U/ml penicillin-streptomycin (Invitrogen) and maintained at 37° C. in a 5% CO₂ incubator. Culture media is typically changed every two days. Cells are typically allowed to expand to 80-90% confluency before passaging with 0.05% trypsin-EDT A (Invitrogen) and replating at a 1:3 ratio.

SERA cells can be isolated by live staining and sorting for cells displaying the SSEA3 marker at high levels, for example, as described in detail in the Examples. Briefly, for example, cells are trypsinized and washed twice with ice cold PBS+2% goat serum (PBS,G). Cells are then passed through a filter (for example, a 40 μm filter) to remove clumps. Following washes, cells are resuspended n ice-cold PBS-G containing 1:200 SSEA3 antibody (Millipore. mab4303) and incubated for 45 min in the dark at 4° C. with gentle rocking After primary antibody binding, cells are washed, for example, three times with ice-cold PBS-G and then resuspended in 1 ml ice-cold PBS-G containing 1:200 Alexa 488-conjugated goat anti-rat IgM (Invitrogen, A21212) and incubated for 45 minutes in the dark at 4° C. with gentle rocking. After secondary antibody binding, cells are washed, for example, three times with ice-cold PBS-G, resuspended in 2 ml of ice-cold PBS-G, passed through a filter (for example, a 40 μm filter), and immediately analyzed and sorted on a cell sorter (for example, a FACSAria cell sorter (BD Biosciences)). For double staining analysis, 1:200 rat anti-human SSEA3 (Millipore, mab4303) and 1:50 mouse anti-human CD105 (BD Biosciences, 611314) primary antibodies can be used in conjunction with 1:200 DyLight 649-conjugated goat anti-rat (Jackson ImmunoResearch, 112-496-075) and FITC-conjugated goat anti-mouse (Invitrogen, M301 01) secondary antibodies respectively. Techniques for labeling, sorting, fluorescence activated cell sorting (FACS), and enrichment of cells are known. Useful examples are described in WO 2001/022507 and U.S. application Ser. No. 13/391,251 (US 2012-0220030 A1), which are hereby incorporated by reference in their entirety, and specifically for their description of cell labeling, sorting, and enrichment.

SSEA3-expressing human skin cells co-express the endoglin (CD105) epitope, as detected using antibody CD105 clone 266 (BD Bioscience, 560839) (see, for example, Kuroda et al. (2010) Proc. Natl. Acad. Sci., U.S.A., 107: 8639-8643; Wakao et al. (2011) Proc. Natl. Acad. Sci., U.S.A., 108: 9875-9880). These SSEA3/CD105 clone 266 identified cells are referred to as multipotent stress enduring (MUSE) cells in reference to their multilineage differentiation capacity (Id.).

However, it has now been discovered that particularly useful SSEA3 cells co express CD105 clone 35 (BD Biosciences, 611314). Investigation of the percent of antibody bound cells using either CD105 antibody clones 266 and 35 showed that CD105 clone 266 bound to almost 100% of primary cultured human skin cells, however, CD105 clone 35 (CD105c35) bound to only around 1% primary cultured human skin cells. Without being bound to a particular theory, it is believed that that CD105c35 binds a rarer variant of endoglin (CD105) while the clone 266 antibody binds an endoglin variant ubiquitously expressed on human primary skin cells. Interestingly, CD105c35 only bound to about 1% of bone marrow derived MSCs, suggesting that this antibody is not an MSC marker.

Thus, it has been discovered that the cells isolated using CD105 clone 35 (BD Biosciences, 611314) represent a rarer and previously unidentified cell type (subpopulation). Further, it has been discovered that this cell type is involved in the repair processes in response to wounding.

A bank of HUF1 cells can be used to establish SSEA3^{NEGATIVE}, SSEA3^{LOW}, and SSEA3^{HIGH} threshold levels for consistent categorization of cell types. An example of this is described herein in the Examples. For example, the top 10% of cells with the highest level of SSEA3 expression can be sorted as representatives for SSEA3^{HIGH} subpopulation and the bottom 10% of cells with the lowest level of SSEA3 expression can be sorted as representatives for the SSEA3^{NEGATIVE} subpopulation. This can be accomplished by, for example, running a standardized control sample (HUF1, as described by Byrne et al. (2009) PLoS ONE, 4: e7118) and setting the gates accordingly (specifically, 13% SSEA3-high, 33% SSEA3-low and 54% SSEA3-negative).

CD146-MSCs, CD271-MSCs and SERA cells can be separated from an initial population of somatic cells ex vivo or in vitro, that is, outside the body of the individual, and sometimes in culture. This initial population of somatic cells is often a complex mixture or a heterogeneous culture of somatic cells. The initial population can be obtained from any animal species, preferably a mammalian species, for example, human, primate, equine, bovine, porcine, canine, feline, etc. The initial population can include fresh or frozen cells, which can be from a neonate, a juvenile or an adult, and from tissues including skin, muscle, bone marrow, peripheral blood, umbilical cord blood, spleen, liver, pancreas, lung, intestine, stomach, and other differentiated tissues. The tissue can be obtained by biopsy or aphoresis from a live donor, or obtained from a dead or dying donor within about 48 hours of death, or freshly frozen tissue, tissue frozen within about 12 hours of death and maintained at below about −20° C., usually at about liquid nitrogen temperature (−190° C.) indefinitely. For isolation of cells from tissue, an appropriate solution can be used for dispersion or suspension of the cells. Such solution can generally be a balanced salt solution, for example, normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

CD146-MSCs, CD271-MSCs and/or SERA cells can be separated from the initial population of somatic cells immediately following dispersion or suspension of the cells. The initial population can be cultured first to form a heterogeneous culture of cells, for example, a primary culture of skin cells, which can then be subjected to separation techniques that can enrich for cells that express cell markers of interest, such as CD 146, CD271, SSEA3, and CD105 (clone 35). The initial population can be frozen and stored frozen, usually at about −80° C. to about liquid nitrogen temperature (−190° C.), until a time at which the separation of CD146-MSCs, CD271-MSCs and/or SERA cells from the initial population can be performed. In such cases, the cells can usually be stored in 10% DMSO, 50% serum, 40% buffered medium, or some other such solution as is commonly used in the art to preserve cells at such temperatures, and can be thawed and recultured by methods commonly known in the art and as described further below.

Separation of CD146-MSCs, CD271-MSCs and/or SERA cells from the initial population of somatic cells can be by any convenient separation technique. For example, CD146-MSCs, CD271-MSCs and/or SERA cells can be separated from the initial population by affinity separation techniques. Techniques for affinity separation can include magnetic separation using magnetic beads coated with an affinity reagent, affinity chromatography, “panning” with an affinity reagent attached to a solid matrix (for example, a plate), cytotoxic agents joined to an affinity reagent or used in conjunction with an affinity reagent (for example, complement and cytotoxins), or other convenient technique. Techniques providing accurate separation include fluorescence activated cell sorters, which can have varying degrees of sophistication, such as multiple color channels, low angle and obtuse light scattering detecting channels, impedance channels, etc. The cells can be selected against dead cells by employing dyes associated with dead cells (for example, propidium iodide). Any technique can be employed that is not unduly detrimental to the viability of CD146-MSCs, CD271-MSCs and/or SERA cells.

To separate CD146-MSCs, CD271-MSCs and/or SERA cells by affinity separation techniques, the initial population of somatic cells can be contacted with an affinity reagent that specifically recognizes and selectively binds the marker(s) of interest, such as CD146, CD271, SSEA3, and CD105 (clone 35). For example, SERA cells can be identified, separated, and/or enriched by identifying, selecting, sorting, etc. cells positive for both SSEA3 and CD105 (clone 35). As another example, CD146-MSCs, CD271-MSCs can be identified, separated, and/or enriched by identifying, selecting, sorting, etc. cells positive for CD146 and CD271.

The affinity reagent can be an antibody, that is, an antibody that is specific for the marker of interest, such as CD146, CD271, SSEA3, and CD105 (clone 35). The affinity reagent can be a specific receptor or ligand for the cell marker, for example, a peptide ligand and receptor; effector and receptor molecules, a T-cell receptor specific for the marker, and the like. Multiple affinity reagents specific for the same marker can be used. Antibodies and T cell receptors can be monoclonal or polyclonal, and can be produced by, for example, transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art. Of particular interest is the use of antibodies as affinity reagents. These antibodies can be conjugated with a label for use in separation. Labels include magnetic beads, which allow for direct separation; biotin, which can be removed with avidin or streptavidin bound to a support; fluorochromes, which can be used with a fluorescence activated cell sorter; or the like, to allow for ease of separation of the particular cell type. Examples of fluorochromes that find use include phycobiliproteins, for example, phycoerythrin and allophycocyanins, fluorescein and Texas red. Each antibody can be labeled with a different fluorochrome, to permit independent sorting for each marker.

The initial population of somatic cells can be contacted with the affinity reagent(s) and incubated or a period of time sufficient to bind the available cell surface antigens. The incubation can usually be at least about 5 minutes and usually less than about 60 minutes. It is desirable to have a sufficient concentration of antibodies in the reaction mixture, such that the efficiency of the separation is not limited by lack of antibody. The appropriate concentration can be determined by titration, but can typically be a dilution of antibody into the volume of the cell suspension that can be about 1:50 (that is, 1 part antibody to 50 parts reaction volume), about 1:100, about 1:150, about 1:200, about 1:250, about 1:500, about 1:1000, about 1:2000, or about 1:5000. The medium in which the cells are suspended can be any medium that maintains the viability of the cells. A preferred medium is phosphate buffered saline containing from 0.1 to 0.5% BSA or 1-4% goat serum. Various media are commercially available and can be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, goat serum etc.

The cells that become labeled by the affinity reagent can be selected for by any convenient affinity separation technique, for example, as described herein or as known in the art. Following separation, the separated cells can be collected in any appropriate medium that maintains the viability of the cells, usually having a cushion of serum at the bottom of the collection tube. Various media are commercially available and can be used according to the nature of the cells, including dMEM, HBSS, dPBS, RPMI, Iscove's medium, etc., frequently supplemented with fetal calf serum.

Compositions highly enriched for CD146-MSCs, CD271-MSCs and/or SERA cells can be achieved in this manner. The CD146-MSCs, CD271-MSCs and/or SERA cells can be about 70%, about 75%, about 80%, about 85% about 90% or more of the cells, about 95% or more of the enriched cells, and can preferably be about 95% or more of the enriched cells. In other words, the composition can be a substantially pure composition of CD146-MSCs, CD271-MSCs and/or SERA cells. The cells of the substantially pure composition can also express higher levels of the gene Nanog than the cells that express no or low levels of SSEA3 from which they were separated. Additionally, the enriched cells can be morphologically indistinguishable from the cells from which they were separated; for example, if enriched from a human dermal fibroblast population, SERA cells can appear morphologically substantially the same as or identical to SSEA3 human dermal fibroblasts.

The cells can be identified, separated, and/or enriched based on cell markers. Generally, whether a cell expresses or displays a cell marker can be expressed as a level of expression or, more commonly, as the cell being positive or negative for the marker. It will be understood by those of skill in the art that the stated expression levels reflect detectable amounts of the marker protein on the cell surface. Generally, cell markers can be assessed by staining or labeling cells with probes that specifically bind the marker of interest and that generate a detectable signal. A cell that is negative for staining (the level of binding of a marker specific reagent is not detectably different from an isotype matched control) can still express minor amounts of the marker. While it is commonplace in the art to refer to cells as “positive” or “negative” for a particular marker, actual expression levels are a quantitative trait. The number of molecules on the cell surface can vary by several logs, yet still be characterized as “positive.”

The staining intensity of cells can be monitored by flow cytometry, where lasers detect the quantitative levels of fluorochrome (which is proportional to the amount of cell surface marker bound by specific reagents, for example, antibodies). Flow cytometry or fluorescence activated cell sorting (FACS, a form of flow cytometry), can also be used to separate cell populations based on the intensity of binding to a specific reagent, as well as other parameters such as cell size and light scatter. Although the absolute level of staining can differ with a particular fluorochrome and reagent preparation, the data can be normalized to a control.

To normalize the distribution to a control, each cell can be recorded as a data point having a particular intensity of staining. These data points can be displayed according to a log scale, where the unit of measure can be arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining brighter than that of an isotype matched control, but is not as intense as the most brightly staining cells normally found in the population. An alternative control can utilize a substrate having a defined density of marker on its surface, for example a fabricated bead or cell line, which provides the positive control for intensity.

The CD146-MSCs, CD271-MSCs and SERA cells can be used immediately. Alternatively, CD146-MSCs, CD271-MSCs and SERA cells can be frozen at liquid nitrogen temperatures and stored for long periods of time. The cells can later be thawed and are capable of being reused. In such cases, the cells can be frozen in, for example, 10% DMSO, 50% serum, 40% buffered medium, or some other such solution as is commonly used in the art to preserve cells at such freezing temperatures. The cells can be thawed in a manner as commonly known in the art for thawing frozen cultured cells.

CD146-MSCs, CD271-MSCs and/or SERA cells can be cultured in vitro under various culture conditions. Culture medium can be liquid or semi-solid, for example, containing agar, methylcellulose, etc. The cell population can be conveniently suspended in an appropriate nutrient medium, such as Iscove's modified DMEM or RPMI-1640, normally supplemented with fetal calf serum (about 5-10%), L-glutamine, a thiol, particularly 2-mercaptoethanol, and antibiotics, for example, penicillin and streptomycin. The cells can be grown to confluence before use. For example, CD271-MSCs can be grown to confluence before treating the cells to induce differentiation (to HOD-MSCs, for example).

The culture can contain growth factors to which the cells are responsive. Growth factors, as defined herein, ace molecules capable of promoting survival, growth and/or differentiation of cells, either in culture or in the intact tissue, through specific effects on a transmembrane receptor. Growth factors include polypeptides and non-polypeptide factors.

CD146-MSCs, CD271-MSCs and/or SERA cells can be used to produce induced pluripotent stem cells (iPSCs) for any purpose and in any technique suitable for pluripotent stem cells, such as embryonic stem cells (ESCs), MSCs, marrow adherent stromal cells (MAPCs), MIAMI, hematopoietic stem cells (HSCs), and especially uses particularly suited to iPSCs, such as for autologous treatments.

The isolated cells can be maintained using standard culture conditions, for example, as described in the Examples.

SERA cells, CD146-MSCs and/or CD271-MSCs can be grown, stored and otherwise maintained over a number of passages. The cells can be high passage cells, with Passage (P) equal or greater than 8. The cells can be low passage cells with P<8, or P<7, or P<6, or P<5, or P<4. The cells can be maintained in culture for less than about 6 weeks, or for less than about 1 month, or for less than about 2 weeks, or for about 1 week or less. The cells can be derived from adult human skin.

### V. Methods of Using CD146-MSCs, CD271-MSCs and SERA Cells

SERA cells, CD146-MSCs and CD271-MSCs can be used directly, such as for therapeutic, research, or other purposes, or can be used to generate derivative cells that can be used for therapeutic, research, or other purposes. For example, iPS cells, differentiated mesenchymal stem cells, and differentiated cells can be derived from SERA cells, CD146-MSCs and/or CD271-MSCs. To support these uses, SERA cells, CD146-MSCs, CD271-MSCs, and/or cells derived from the SERA cells, CD146-MSCs, CD271-MSCs, or both can be stored, banked, proliferated, cultured, etc. for eventual use in therapy, research, and other purposes. The cells can be used as or to produce cells that are suitable for in vivo methods and/or ex vivo methods and/or in vitro methods.

The CD146-MSCs and CD271-MSCs can be used for any purpose and in any way that MSCs can be used. For example, CD146-MSCs and CD271-MSCs can be cultured and then differentiated to various types of multipotent mesenchymal stem cells, such as human osteogenic dermal mesenchymal stem cells (HOD-MSCs), human chondrogenic dermal mesenchymal stem cells (HCD-MSCs), and human adipogenic dermal mesenchymal stem cells (HAD-MSCs). Thus, disclosed are methods of generating differentiated multipotent mesenchymal stem cells from CD146-MSCs and CD271-MSCs. Such differentiated MSCs can be used for therapeutic, research, and other purposes. Thus, provided herein are methods of treatment using differentiated MSCs and cells differentiated from differentiated MSCs.

CD146-MSCs and CD271-MSCs can be differentiated to various types of cells, such as osteoblasts, osteocytes, chondroblasts, chondrocytes, cardiomyoblasts, cardiomyocytes, neuroblasts, neurocytes, pancreocytes, hepatoblasts, hepatocytes, and hematopoietic stem cells. Thus, disclosed are methods of generating cells differentiated from CD146-MSCs and CD271-MSCs. Such differentiated cells can be used for therapeutic, research, and other purposes. Thus, disclosed are methods of treatment using cells differentiated from CD146-MSCs and CD271-MSCs. Also disclosed are cell lines and cell strains generated from proliferation of CD146-MSCs and CD271-MSCs ex vivo from skin dermis.

CD146-MSCs and CD271-MSCs can differentiate into adipocytes, osteoblasts, or chondrocytes under conditions that induce such differentiation. CD146-MSCs and CD271-MSCs can be isolated from an in vitro culture of primary mammalian skin cells. CD146-MSCs and CD271-MSCs can express one or markers selected from the group consisting of CD10, CD13, CD26, CD29, CD34, CD44, CD54, CD71, CD73, CD90, CD105 (clone 266), CD106, CD166, ITGA11, STRO-1 and SSEA4.

Disclosed are methods of treating a mammal that involve applying or administering to the mammal SERA cells, CD146-MSCs and CD271-MSCs, and/or cells derived from the SERA cells, CD146-MSCs, CD271-MSCs, or all three. For example, the methods can involve applying or administering to a mammal a cell line or cell strain derived from SERA cells, CD146-MSCs or CD271-MSCs. The methods can involve applying or administering to a mammal any of the disclosed therapeutic compositions.

SERA cells in particular are useful for direct use in therapy since SERA cells are associated with skin regeneration. Thus, SERA cells can be used directly in methods and devices for regenerating, repairing, replacing, and supporting skin. CD146-MSCs and CD271-MSCs, being stem cells, are particularly useful for generating other cells, such as iPS cells, differentiated mesenchymal stem cells, and differentiated cells.

SERA cells, CD146-MSCs CD271-MSCs, and/or cells derived from the SERA cells, CD146-MSCs, CD271-MSCs, or all three, and devices having SERA cells, CD146-MSCs, CD271-MSCs, and/or cells derived from the SERA cells, CD146-MSCs, CD271-MSCs, or all three can be used to treat or repair damaged or missing tissue. Tissues that can be treated include but are not limited to bone, cartilage, heart, dental, cardiovascular, skin, liver, neural, or kidney tissue. For example, grafts having SERA cells or cells derived from the SERA cells, CD146-MSCs, CD271-MSCs, or all three can be used to repair or regenerate skin. Composition containing cardiac cells derived from CD 146-MSCs or CD271-MSCs can be injected or implanted into cardiac tissue. Implants containing cells derived from CD146-MSCs or CD271-MSCs can be used to repair fractured or broken bones, to fuse or replace bones, or to improve the integration of an implant into existing bone tissue. Dental implants can be used to treat or repair damaged or missing teeth, and facial bones. The dental implant can be entirely for aesthetic purposes. Implants can also be used as a filler to augment or form tissue or to support the function of natural tissues or prosthesis. For example, compositions containing SERA cells can be used as cosmetic fillers. The device can be configured for implantation into the target tissue, such as skin, bone, cartilage, heart, dental, cardiovascular, skin, liver, neural, or kidney tissue.

The devices and implants can be used to treat bone disorders including, but not limited to, bone fractures, bone degeneration, osteoporosis, broken bones, spinal injuries, chipped bones, herniated vertebral discs, skull fractures, etc. The devices can also be used to treat dental disease including dental diseases that cause loss of teeth or loss of bone.

Differentiated cells derived from the cells can be terminally differentiated cells, or they can be capable of giving rise to cells of a specific lineage. For example, the cells can be differentiated into a variety of multipotent cell types, for example, neural stem cells, cardiac stem cells, or hepatic stem cells. The stem cells can then be further differentiated into new cell types, for example, neural stem cells can be differentiated into neurons; cardiac stem cells can be differentiated into cardiomyocytes; and hepatic stem cells can be differentiated into hepatocytes.

iPS cells can be used for reconstituting or supplementing differentiating or differentiated cells in a subject. The induced cells can be differentiated into cell-types of various lineages. Examples of differentiated cells include any differentiated cells from ectodermal (for example, neurons and fibroblasts), mesodermal (for example, cardiomyocytes), or endodermal (for example, pancreatic cells) lineages. The differentiated cells can be, for example, pancreatic beta cells, neural stem cells, neurons (for example, dopaminergic neurons), oligodendrocytes, oligodendrocyte progenitor cells, hepatocytes, hepatic stem cells, astrocytes, myocytes, hematopoietic cells, or cardiomyocytes.

SERA cells, CD146-MSCs, CD271-MSCs, and/or cells derived from the SERA cells, CD146-MSCs and/or CD271-MSCs, including cells differentiated from SERA cells, CD146-MSCs and/or CD271-MSCs, can be used as or in therapy to treat disease (for example, a genetic defect). The therapy can be directed at treating the cause of the disease; or alternatively, the therapy can be to treat the effects of the disease or condition. For example, the cells can be transferred to, or close to, an injured site in a subject; or the cells can be introduced to the subject in a manner allowing the cells to migrate, or home, to the injured site. The transferred cells can advantageously replace the damaged or injured cells and allow improvement in the overall condition of the subject. The transferred cells can stimulate tissue regeneration or repair. Transferred cells can be cells differentiated from the disclosed cells. The transferred cells also can be multipotent stem cells differentiated from the disclosed cells. In some cases, the transferred cells can be disclosed cells that have not been differentiated.

SERA cells, CD146-MSCs and CD271-MSCs, and/or cells derived from the SERA cells, CD146-MSCs and/or CD271-MSCs, including cells differentiated from SERA cells, CD146-MSCs and/or CD271-MSCs, can be used for cosmetic effects. For example, the cells can be used in skin fillers to smooth or adjust skin profile. The cells can also be used to augment cartilage to alter skin profile.

The number of administrations of treatment to a subject can vary. Introducing SERA cells, CD146-MSCs, CD271-MSCs, and/or cells derived from the SERA cells, CD146-MSCs and/or CD271-MSCs, such as differentiated cells, into the subject can be a one-time event. Alternatively, such treatment may elicit improvement for only a limited time, which would indicate an on-going series of repeated treatments. In other situations, multiple administrations of the cells may be needed before an effect is observed. The exact protocols depend upon the disease or condition, the stage of the disease and parameters of the individual subject being treated.

The cells can be introduced to the subject via any suitable routes. For example, the cells can be introduced to the subject via parenteral, intravenous, intraarterial, intramuscular, subcutaneous, transdermal, intratracheal, intraperitoneal, or into spinal fluid. The cells can be implanted, transplanted, applied, etc. The route of administration can be selected based on the disease or condition, the effect desired, and the nature of the cells being used.

CD146-MSCs, CD271-MSCs, iPSCs, and/or reprogrammed SERA cells can be differentiated. Differentiated cells can be used for any purpose and in any manner for which such differentiated cells can be put. Such uses generally depend on the type of differentiated cell that is produced. Many such uses are known, some of which are described herein. Differentiated cells can be administered, implanted, or transferred to subjects suffering from a wide range of diseases or disorders. Subjects suffering from neurological diseases or disorders could especially benefit from stem cell therapies. In some approaches, the cells can be differentiated into neural stem cells or neural cells and then transplanted to an injured site to treat a neurological condition (for example, Alzheimer's disease, Parkinson's disease, multiple sclerosis, cerebral infarction, spinal cord injury, or other central nervous system disorder; see, for example, Morizane et al. (2008) Cell Tissue Res., 331(1): 323-326; Coutts and Keirstead (2008) Exp. Neurol., 209(2): 368-377; Goswami and Rao (2007) Drugs, 10(10): 713-719).

For the treatment of Parkinson's disease, the cells can be differentiated into dopamine-acting neurons and then transplanted into the striate body of a subject with Parkinson's disease. For the treatment of multiple sclerosis, the cells can be differentiated into oligodendrocytes or progenitors of oligodendrocytes, which are then transferred to a subject suffering from MS.

For the treatment of any neurologic disease or disorder, a successful approach can be to introduce neural stem cells to the subject. For example, in order to treat Alzheimer's disease, cerebral infarction or a spinal injury, the cells can be differentiated into neural stem cells followed by transplantation into the injured site. The cells can also be engineered to respond to cues that can target their migration into lesions for brain and spinal cord repair (for example, Chen et al. (2007) Stem Cell Rev., 3(4): 280-288).

Diseases other than neurological disorders can also be treated by a stem cell therapy that uses cells differentiated from disclosed cells, such as multipotent or pluripotent stem cells. Degenerative heart diseases such as ischemic cardiomyopathy, conduction disease, and congenital defects can benefit from stem cell therapies (see, for example, Janssens et al. (2006) Lancet, 367: 113-121).

Pancreatic islet cells (or primary cells of the islets of Langerhans) can be transplanted into a subject suffering from diabetes (for example, diabetes mellitus, type 1) (see for example, Burns et al. (2008) Curr. Stem Cell Res. Ther., 2: 255-266). Thus, pancreatic beta cells derived from the disclosed cells can be transplanted into a subject suffering from diabetes (for example, diabetes mellitus, type 1).

Hepatic cells or hepatic stem cells derived from the disclosed cells can be transplanted into a subject suffering from a liver disease, for example, hepatitis, cirrhosis, or liver failure.

Hematopoietic cells or hematopoietic stem cells (HSCs) derived from the disclosed cells can be transplanted into a subject suffering from cancer of the blood, or other blood or immune disorder. Examples of cancers of the blood that are potentially treated by hematopoietic cells or HSCs include: acute lymphoblastic leukemia, acute myeloblastic leukemia, chronic myelogenous leukemia (CML), Hodgkin's disease, multiple myeloma, and non-Hodgkin's lymphoma. Often, a subject suffering from such disease must undergo radiation and/or chemotherapeutic treatment in order to kill rapidly dividing blood cells. Introducing HSCs derived from the disclosed cells to these subjects can help to repopulate depleted reservoirs of cells.

Hematopoietic cells or HSCs derived from the disclosed cells can also be used to directly fight cancer. For example, transplantation of allogeneic HSCs has shown promise in the treatment of kidney cancer (see, for example, Childs et al. (2000) N. Engl. J. Med., 343: 750-758). Allogeneic, or even autologous, HSCs derived from the disclosed cells can be introduced into a subject in order to treat kidney or other cancers.

Hematopoietic cells or HSCs derived from the disclosed cells can also be introduced into a subject in order to generate or repair cells or tissue other than blood cells, for example, muscle, blood vessels, or bone. Such treatments can be useful for a multitude of disorders.

Cells and cell lines can also be produced using animals. For example, SERA cells, CD146-MSCs, CD271-MSCs, and/or cells derived from the SERA cells, CD146-MSCs and/or CD271-MSCs can be transferred into an immunocompromised animal, for example, SCID mouse, and allowed to differentiate. The transplanted cells can form a mixture of differentiated cell types and tumor cells. The specific differentiated cell types of interest can be selected and purified away from the tumor cells by use of lineage specific markers, for example, by fluorescent activated cell sorting (FACS) or other sorting method, for example, magnetic activated cell sorting (MACS). The differentiated cells can then be transplanted into a subject (for example, an autologous subject, HLA-matched subject) to treat a disease or condition. The disease or condition can be, for example, a hematopoietic disorder, an endocrine deficiency, degenerative neurologic disorder, hair loss, or other disease or condition.

The cells can be used in methods for identifying and optimizing conditions to differentiate stem cells. The process of differentiation can proceed in a stepwise fashion with cells progressing from one precursor cell to the next before their final cell type. An example can be found in the hematopoietic system where the primordial stem cell gives rise to various precursors which in turn generate additional precursors before the appearance of the final B cell or T cell. Disclosed are methods and compositions that can be used to define this progression, or any other, from precursor to final product, and include the disclosed reversible transformation system.

The cells can also be used for toxicology testing. For example, ACTIVTox, based on a human liver cell line, is designed to provide a high throughput, metabolically active platform for the development of structure toxicity relationships. The disclosed cells can be used in analogous systems for toxicology testing.

SERA cells and/or cells derived from the SERA cells, CD146-MSCs, CD271-MSCs, or all three can be used in the treatment of wounds. Such wounds include, but are not limited to lacerations and/or abrasions, surgical wounds, amputations, burns, chronic diabetic wounds, and the like. The cells can be applied directly to a wound site. The cells can be incorporated into natural or artificial grafts.

A. Direct Use in Wound Repair

The cells (for example, the SERA cells, CD146-MSCs and/or CD271-MSCs, preferably derived from the subject into which they are to be implanted) can be directly injected or placed into a site in need of repair. This may be the preferred method of treatment since vascular delivery suffers from a “pulmonary first pass effect” where intravenous injected cells are often sequestered in the lungs. Accordingly, the cells can be applied directly to the wound site or injected into tissue constituting or surrounding the wound.

The cells can be provided in a matrix formulation or scaffold to facilitate cell survival, maintenance, and/or growth. For example, the cells can be provided in a fibrin scaffold as described, for example, in U.S. Patent Publication No: 20120039855. As described therein, a fibrin scaffold is provided that is capable of supporting or anchoring embedded MSC or other (for example, SERA cells) to an implantation site for a therapeutically effective period of time. In particular, the fibrin scaffold described therein facilitates blood vessel formation at a given implantation site of a subject. The fibrin scaffold can be formed with a fibrinogen component containing fibrinogen having a final concentration of higher than 17.5 mg/ml scaffold. In particular, the fibrin scaffold can be capable of supporting a population of mesenchymal stem cells (MSC) and/or other derived cells seeded to a concentration of at least 1×10⁶ cells/ml scaffold. The scaffold can also have a biologically active component, For example, the scaffold can include a solution of proteins derived from blood plasma that can also have anti fibrinolytic agents such as tranexamic acid and/or stabilizers such as arginine, lysine, their pharmaceutically acceptable salts, or mixtures thereof. The solution can have additional factors such as, for example, factor VIII, fibronectin, von Willebrand factor (vWF), vitronectin, etc. Examples of this are described in U.S. Pat. No. 6,121,232 and WO 9833533. The solution can also have stabilizers such as tranexamic acid and arginine hydrochloride.

Any other suitable scaffold/matrix materials can be utilized to contain and apply the disclosed cells to a wound site. Such scaffold/matrix materials can have a natural or synthetic cell extracellular matrix material (ECM). Such extracellular matrix materials are well known to those of skill in the art (see, for example, Halstenberg et al. (2002) Biomacromolecules 3: 710-723; Mann et al. (2001) Biomaterials 22: 3045-3051, and the like). Illustrative synthetic ECM materials include, for example, hydrogel ECMs formed from biological materials (for example, hyaluronic and collagen hydrogels, see, for example, HYSTEM® hydrogels) or ECMs formed from synthetic hydrogel materials (for example, PEG-tetravinylsulfone, see, for example, Lutolf et al. (2003) Proc. Natl. Acad. Sci., USA, 100(9): 5413-5418, and the like).

The foregoing scaffold/matrix materials are intended to be illustrative and not limiting. Using the teaching provided herein, numerous other scaffold materials suitable of application of SERS and/or MSCs described herein to a wound site will be available to one of skill in the art.

B. Use in Synthetic Grafts or Augmented Skin Grafts

The SERA cells and/or cells derived from SERA cells, CD146-MSCs and/or CD271-MSCs can be incorporated into synthetic or natural graft materials.

1. Synthetic Grafts

SERA cells and/or cells derived from SERA cells, CD146-MSCs and/or CD271-MSCs can be incorporated into a synthetic graft material. Synthetic skin (and other tissue) graft materials are known to those of skill in the art. Illustrative synthetic skin materials include, but are not limited to, DERMAGRAFT®, INTEGRA® artificial skin, and BIOBRANE®. BIOBRANE® is a nylon material that contains a gelatin that interacts with clotting factors in the wound. That interaction causes the dressing to adhere better, forming a more durable protective layer. Integra is a two-layered dressing. The top layer serves as a temporary synthetic epidermis; the layer below serves as a foundation for regrowth of dermal tissue. The underlying layer is made of collagen fibers that act as a lattice through which the body can begin to align cells to recreate its own dermal tissue.

SERA cells and/or cells derived from SERA cells, CD146-MSCs and/or CD271-MSCs can be integrated into (for example, seeded into) the synthetic skin, for example, DERMAGRAFT®, BIOBRANE®, INTEGRA®, and the like.

More generally, synthetic skins are contemplated that include a scaffold and a therapeutic component. The scaffold typically can be a matrix of fibers that forms interstices. The therapeutic component can include a cellular component and, optionally, a non-cellular component. The cellular component can include, for example, SERA cells and/or cells derived from the SERA cells, CD146-MSCs, CD271-MSCs, or all three. The fiber material can be a synthetic structural material such as a biocompatible polymer. Biodegradable polymers are also desirable. Such materials can be selected such that they are dissolved or resorbed by the body without the need for surgical removal procedures. Biocompatible, biodegradable materials useful in the grafts include, but are not limited to, polyglycolic acid (PGA), type 1 collagen, Poly-DL-lactide-caprolactone (PCL), laminin, gelatin, and the like. The non-cellular component can include biologically active agents in addition to those secreted by the cells, for example, hormones, steroids, anti-inflammatories, analgesics, immunosuppressives, anticoagulants, muscle relaxants and antispasmodics, antibiotics and/or antimicrobials, growth factors, colony stimulating factors, nutrients such as vitamins, peptides, small drug molecules, gene therapy agents, for example, plasmids, retrovirals, and combinations thereof; and other pharmacologically acceptable excipients, diluents, buffers, preservatives, and the like. Methods of making artificial skins are described in U.S. Patent Publication Nos: 2012/0015022, 2011/0318414, 2011/0052693, 2009/0232878, 2008/0311613, 2008/0248571, 2008/0033550, 2004/0162615, 2004/0038859, 2003/0109927, 2002/0193875, 2002/0082692, which are incorporated herein by reference for the artificial skins and synthetic methods described therein.

2. Augmented Skin Grafts

The cells can be incorporated into natural skin grafts to form cell augmented grafts. Methods of preparing skin grafts are well known to those of skill in the art. The skin graft can be an autologous skin graft (autograft) where the donor skin is taken from a different site on the same individual's body (also known as an autograft). The skin graft can be isogenic where the donor and recipient individuals are genetically identical (for example, monozygotic twins, animals of a single inbred strain). The graft can be allogeneic (the donor and recipient are of the same species) and the graft can be obtained from a live donor or from a cadaver.

The cells can be applied to a wound at the time the graft is applied to the subject. The cells can be integrated into the graft material (for example, seeded into the graft) prior to application to the recipient.

The foregoing methods of use and graft materials are intended to be illustrative and non-limiting. Using the teachings provided herein, numerous applications of the SERA cells and/or cells derived from SERA cells, CD146-MSCs and/or CD271-MSCs described herein will be available to one of skill in the art.

The dosages or amounts of the cells, devices, compositions, or compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The efficacy of a particular dose of the cells, devices, compositions, or compounds described herein can be determined by evaluating the particular aspects of the medical history, signs, symptoms, and objective laboratory tests that are known to be useful in evaluating the status of a subject in need treatment for a given disease or condition. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual, the criterion could be (1) a subject's physical condition is shown to be improved, (2) the progression of the disease or condition is shown to be stabilized, or slowed, or reversed, or (3) the need for other medications for treating the disease or condition is lessened or obviated, then a particular treatment regimen will be considered efficacious.

For tissue repair or regeneration, the dosages can be effective to repair or regenerate the tissue to some extent, such as addition of at least 10% by volume of the tissue to be repaired or replaced. For cell or tissue transplantation, the dosages can be effective to produce biologically functioning tissue. For example, transplanted skin can provide skin function; transplanted neural cells can provide dopamine producing nerve cells; transplanted cardiac cells can contribute to heart beating; pancreatic islet cells can provide insulin production. The ideal is to provide or restore full biological function of the tissue, but provision of some measurable biological function can be useful, and dosages effective to provide some measurable biological function can be used. These are only examples. Other biological functions to be provided will be relevant to other diseases and conditions.

### VI. Production of Induced Pluripotent Stem Cells

CD146-MSCs, CD271-MSCs and SERA cells can be used to produce iPS cells. To induce the cells to become iPS cells, a substantially pure population of disclosed cells can be contacted with Reprogramming Factors (RFs). Reprogramming factors, as used herein, refers to one or more, that is, a cocktail of, biologically active factors that act on a cell to alter transcription, thereby reprogramming a cell to multipotency or to pluripotency. The reprogramming factor can be a transcription factor, including without limitation, Oct3/4; Sox2; K1f4; c-Myc; Nanog; and Lin-28.

An Oct3/4 polypeptide is a polypeptide including an amino acid sequence that is at least 70% identical to the amino acid sequence of human Oct 3/4, also known as Homo sapiens POU class 5 homeobox 1 (POU5F1) the sequence of which can be found at GenBank Accession Nos. NP_{—}002692 and NM_{—}002701. Oct3/4 polypeptides, for example, those that are at least 70%, 75%, 80%, 85%, 90%, 91%, 92%, 95%, 97%, 99%, or 100% identical to the sequence provided in GenBank Accession No. NM_{—}002701, and the nucleic acids that encode them find use as a reprogramming factor in the disclosed methods.

A Sox2 polypeptide is a polypeptide including an amino acid sequence at least 70% identical to the amino acid sequence of human Sox2, that is, sex-determining region Ybox 2 protein, the sequence of which can be found at GenBank Accession Nos. NP_{—}003097 and NMOO3106. Sox2 polypeptides, for example, those that are at least 70%, 75%, 80%, 85%, 90%, 91%, 92%, 95%, 97%, 99%, or 100% identical to the sequence provided in GenBank Accession No. NM_{—}003106, and the nucleic acids that encode them find use as a reprogramming factor in the disclosed methods.

A Kf4 polypeptide is a polypeptide including an amino acid sequence that is at least 70% identical to the amino acid sequence of human K1t4, that is, Kruppel-Like Factor 4 the sequence of which can be found at GenBank Accession Nos. NP 004226 and NM_{—}004235. KIM polypeptides, for example, those that are at least 70%, 75%, 80%, 85%, 90%, 91%, 92%, 95%, 97%, 99%, or 100% identical to the sequence provided in GenBank Accession No. NM_{—}004235, and the nucleic acids that encode them find use as a reprogramming factor in the disclosed methods.

A c-Myc polypeptide is a polypeptide including an amino acid sequence that is at least 70% identical to the amino acid sequence of human c-Myc, that is, myelocytomatosis viral oncogene homolog, the sequence of which can be found at GenBank Accession Nos. NP_{—}002458 and NM_{—}002467. c-Myc polypeptides, for example, those that are at least 70%, 75%, 80%, 85%, 90%, 91%, 92%, 95%, 97%, 99%, or 100% identical to the sequence provided in GenBank Accession No. NM_{—}002467, and the nucleic acids that encode them find use as a reprogramming factor in the disclosed methods.

A Nanog polypeptide is a polypeptide including an amino acid sequence that is at least 70% identical to the amino acid sequence of human Nanog, that is, Nanog homeobox, the sequence of which can be found at GenBank Accession Nos. NP_{—}079141 and NM_{—}024865. Nanog polypeptides, for example, those that are at least 70%, 75%, 80%, 85%, 90%, 91%, 92%, 95%, 97%, 99%, or 100% identical to the sequence provided in GenBank Accession No. NM_{—}024865, and the nucleic acids that encode them find use as a reprogramming factor in the disclosed methods.

A Lin-28 polypeptide is a polypeptide including an amino acid sequence that is at least 70% identical to the amino acid sequence of human Lin-28, that is, Lin-28 homolog of C. elegans, the sequence of which can be found at GenBank Accession Nos. NP_{—}078950 and NM_{—}024674. Lin-28 polypeptides, for example, those that are at least 70%, 75%, 80%, 85%, 90%, 91%, 92%, 95%, 97%, 99%, or 100% identical to the sequence provided in GenBank Accession No. NM_{—}024674, and the nucleic acids that encode them find use as a reprogramming factor in the disclosed methods.

Reprogramming factors can be provided to the disclosed cells as nucleic acids encoding said reprogramming factors. Many vectors useful for transferring exogenous genes into target mammalian cells are available. The vectors can be maintained episomally, for example, as plasmids, minicircle DNAs, virus-derived vectors such cytomegalovirus, adenovirus, etc., or they can be integrated into the target cell genome, through homologous recombination or random integration, for example, retrovirus derived vectors such as MMLV, HIV-1, ALV, etc.

Nucleic acids encoding the reprogramming factors can be provided directly to the disclosed cells. In other words, SERA cells can be contacted with vectors including nucleic acids encoding the reprogramming factors such that the vectors are taken up by the cells. Methods for contacting cells with nucleic acid vectors, such as electroporation, calcium chloride transfection, and lipofection, are well known in the art.

Alternatively, nucleic acids encoding the reprogramming factors can be provided to the cells by contact with viral particles including nucleic acids encoding the reprogramming factors. Retroviruses, for example, lentiviruses, are particularly suitable to the disclosed methods, as they can be used to transfect non-dividing cells (see, for example, Uchida et al. (1998) Proc. Natl. Acad. Sci. USA, 95(20): 11939-44). Commonly used retroviral vectors are “defective,” that is, unable to produce viral proteins required for productive infection. Rather, replication of the vector requires growth in a packaging cell line.

To generate viral particles including nucleic acids encoding the reprogramming factors, the retroviral nucleic acids including the nucleic acid encoding the reprogramming factors can be packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, for example, MMLV, are capable of infecting most murine and rat cell types, and can be generated by using ecotropic packaging cell lines such as BOSC23 (Pear et al. (1993) Proc. Natl. Acad. Sci. USA, 90: 8392-8396). Retroviruses bearing amphotropic envelope protein, for example, 4070A (Danos et al. supra.), are capable of infecting most mammalian cell types, including human, dog and mouse, and can be generated by using amphotropic packaging cell lines such as PAI2 (Miller et al. (1985) Mol. Cell. Biol. 5: 431-437); PA317 (Miller et al. (1986) Mol. Cell. Biol. 6: 2895-2902); GRIP (Danos et al. (1988) Proc. Natl. Acad. Sci. USA, 85: 6460-6464). Retroviruses packaged with xenotropic envelope protein, for example, AKR env, are capable of infecting most mammalian cell types, except murine cells. The appropriate packaging cell line can be used to ensure that the SERA cells are targeted by the packaged viral particles. Methods of introducing the retroviral vectors including the nucleic acid encoding the reprogramming factors into packaging cell lines and of collecting the viral particles that are generated by the packaging lines are well known in the art.

Vectors used for providing reprogramming factors to the cells typically include suitable promoters for driving the expression, that is, transcriptional activation, of the reprogramming factor nucleic acids. This can include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing reprogramming factors to the cells can include genes that must later be removed, for example, using a recombinase system such as Cre/Lox, or the cells that express them destroyed, for example, by including genes that allow selective toxicity such as herpesvirus TK, bcl-xs, etc.

The reprogramming factors can be provided as nuclear acting, non-integrating polypeptides of the reprogramming factors, or reprogramming factor polypeptides. In other words, the cells can be contacted with polypeptides that encode the reprogramming factors and act in the nucleus. By non-integrating, it is meant that the polypeptides do not integrate into the genome of the host cell, that is, the SERA cells.

Typically, a reprogramming factor polypeptide can contain the polypeptide sequences of the reprogramming factor fused to a polypeptide permeant domain. A number of permeant domains are known in the art and can be used in nuclear acting, non-integrating polypeptides, including peptides, peptidomimetics, and non-peptide carriers. For example, a permeant peptide can be derived from the third alpha helix of Drosophila melanogaster transcription factor Antennapaedia, referred to as penetratin. As another example, the permeant peptide that includes the HIV-1 tat basic region amino acid sequence, which can include, for example, amino acids 49-57 of naturally-occurring tat protein. Other permeant domains include poly-arginine motifs, for example, the region of amino acids 34-56 of HIV-I rev protein, nona-arginine, octa-arginine, and the like (see, for example, Futaki et al. (2003) Curr Protein Pept Sci. 2003 April; 4(2): 87-96; and Wender et al. (2000) Proc. Natl. Acad. Sci. USA, 97(24): 13003-8; published U.S. Patent applications 2003/0220334; 2003/0083256; 2003/0032593; and 2003/0022831, specifically incorporated herein by reference for the teachings of translocation peptides and peptoids). The nona-arginine (R9) sequence is one of the more efficient protein transduction domains (PTDs) that have been characterized (Wender et al. (2000) supra; Uemura et al. (2002) supra).

The reprogramming factor polypeptide sequences of the reprogramming factor polypeptide can optionally also be fused to a polypeptide domain that increases solubility of the product. Usually the domain is linked to the RF through a defined protease cleavage site, for example, a TEV sequence, which is cleaved by TEV protease. The linker can also include one or more flexible sequences, for example, from 1 to 10 glycine residues. The cleavage of the fusion protein can be performed in a buffer that maintains solubility of the product, for example, in the presence of from 0.5 to 2 M urea, in the presence of polypeptides and/or polynucleotides that increase RF solubility, and the like. Domains of interest include endosomolytic domains, for example, influenza HA domain; and other polypeptides that aid in production, for example, IF2 domain, GST domain, GRPE domain, and the like.

The reprogramming factor polypeptides can be generated in a cell based system using methods known in the art. A nucleic acid (for example, cDNA or genomic DNA) encoding the reprogramming factor polypeptide can be inserted into a replicable vector for expression. Many such vectors are available. The vector components generally include, but are not limited to, one or more of the following: an origin of replication, one or more marker genes, an enhancer element, a promoter, and a transcription termination sequence.

Reprogramming factor polypeptides can be produced recombinantly not only directly, but also as a fusion polypeptide with a heterologous polypeptide, for example, a polypeptide having a specific cleavage site at the N-terminus of the mature protein or polypeptide.

### EXAMPLES

The following examples are offered to illustrate, but not to limit the claimed invention.

### Example 1

### Human Skin Cells that Express Stage-Specific Embryonic Antigen 3 Associate with Dermal Tissue Regeneration

Stage-specific embryonic antigen 3 (SSEA3) is a glycosphingolipid that has previously been used to identify cells with stem cell-like, multipotent and pluripotent characteristics. A rare subpopulation of SSEA3 expressing cells exists in the dermis of adult human skin. These SSEA3-expressing cells undergo a significant increase in cell number in response to injury, suggesting a possible role in regeneration. These SSEA3-expressing regeneration-associated (SERA) cells were derived through primary cell culture, purified by fluorescence activated cell sorting (FACS) and characterized. Longer in vitro culture of the primary skin cells led to lower SSEA3 expression stability following FACS-based purification, suggesting that culture conditions can be further optimized to increase the efficiency of large-scale expansion of SERA cells. The SERA cells demonstrated a global transcriptional state most similar to bone marrow and fat derived mesenchymal stem cells (MSCs) and the highest expressing SSEA3 expressing cells co-expressed CD105 (clone 35). However, while a rare population of MSCs was observed in primary human skin cell cultures that could differentiate into adipocytes, osteoblasts or chondrocytes, SERA cells did not possess this differentiation capacity, suggesting that there are at least two different rare subpopulations in adult human skin primary cultures. The identification, efficient purification and large-scale expansion of these rare subpopulations (SERA cells and MSCs) from heterogeneous adult human skin primary cell cultures have applications for patient-specific cellular therapies.

### Material and Methods

In Vitro Tissue Injury/Regeneration-Associated Assay

Two to six biological replicates from each of three skin biopsy donors (with each biological replicate representing a dermal tissue biopsy fragment of at least 1 mm³ in volume) were analyzed for this study. Analysis of biopsy fragment cellular subpopulations was performed through cryosectioning and immunohistochemical staining, as described by Vaculik et al. (2012) J Invest Dermatol, 132: 563-574; Byrne et al. (2009), supra. Briefly, human skin biopsy fragments were placed into an “Optimal Cutting Temperature” (OCT) compound tissue mold, frozen to −80° C., cut into sections approximately 5 μm thick in a cryostat at −20° C. and analyzed via immunohistochemistry for the SSEA3 antibody. In vitro biopsy adhesion, cell migration and extended primary cell culture (one month) in regular cell culture media—consisting of Dulbecco's Modified Eagle Medium Nutrient Mixture F-12 (DMEM/F12) supplemented with 10% fetal bovine serum (FBS, Invitrogen), 1% MEM non-essential amino acids, 2 mM GlutaMAX™ and 100 IU/ml penicillin-streptomycin (Invitrogen)—was used as an in vitro assay for tissue injury. Following one month of primary cell culture, biopsy fragments were manually removed from tissue culture, cryosectioned and analyzed via immunohistochemistry.

In Vitro Culture of Primary Human Skin Cells

The human skin-derived (HUF1) primary cell line used in this study was obtained from a 4 mm adult skin punch biopsy as described by Byrne et al. (2009) supra. All human biopsy derived cells were cultured in DMEM/F12/FBS culture media. Culture media was changed every two days. Cells were allowed to expand to 80-90% confluency before passaging with 0.05% trypsin-EDTA (Invitrogen) and replating at a 1:3 ratio. A large bank of early passage HUF1 cells was cryopreserved in culture media supplemented with 10% dimethyl sulphoxide (DMSO, Fisher). All research adhered to National Academy of Sciences guidelines.

Stage-Specific Embryonic Antigen 3 (SSEA3) Live Cell Staining and Fluorescence Activated Cell Sorting (FACS)-Based Purification

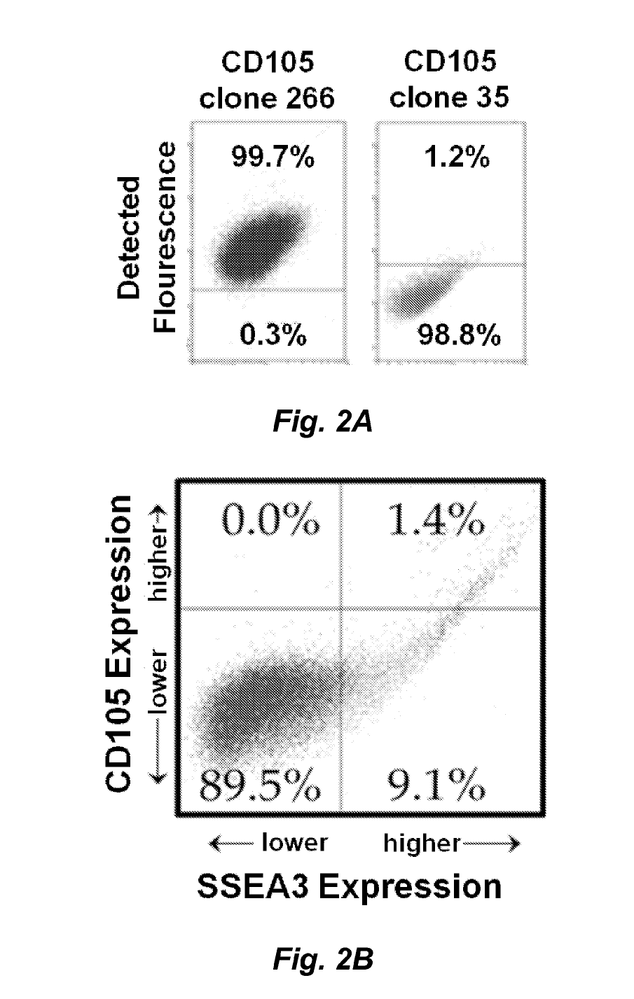

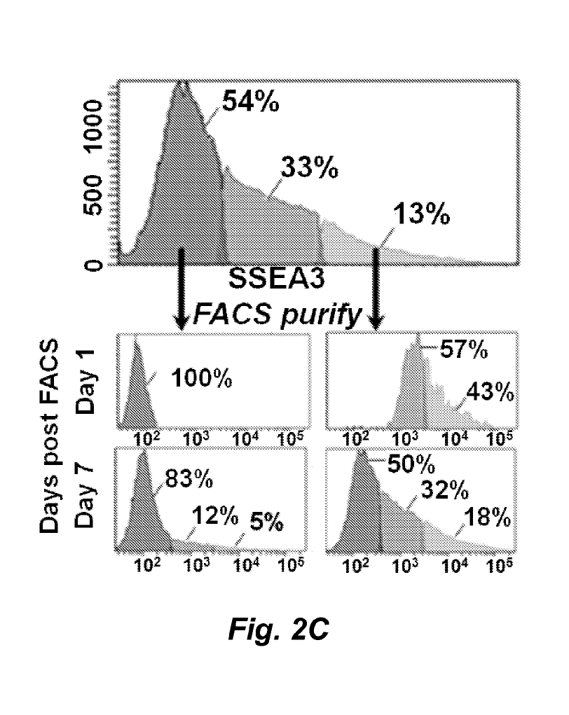

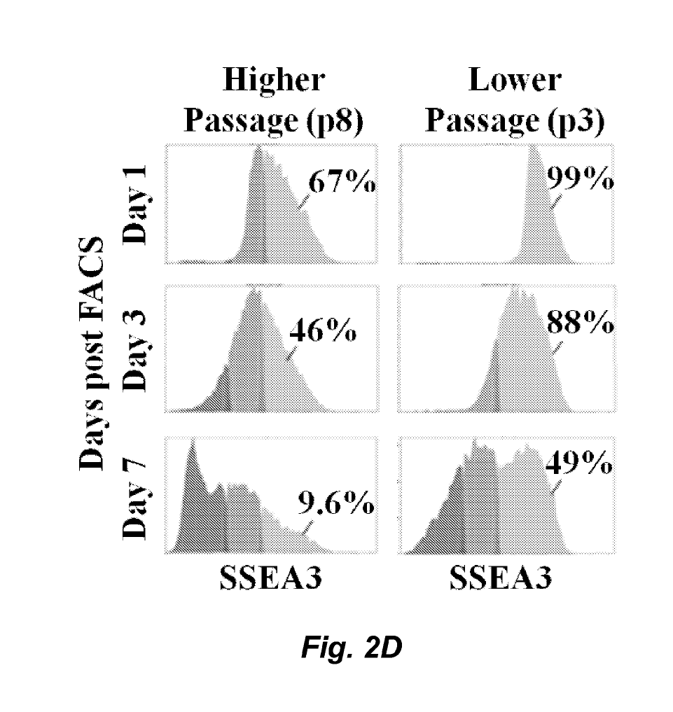

Up to 100 million primary human adherent dermal (PHAD) cells per experiment were trypsinized and washed twice with ice cold PBS+2% goat serum (PBS-G). Cells were then passed through a 40 micrometer filter to remove clumps. Following washes, cells were resuspended in 0.5 ml (per 10 million cells) of ice-cold PBS-G containing 1:100 SSEA3 antibody (Millipore, mab4303) and incubated for 45 min in the dark at 4° C. with gentle rocking After primary antibody binding, cells were washed three times with ice-cold PBS-G and then resuspended in 1 ml ice-cold PBS-G containing 1:200 Alexa 488-conjugated goat anti-rat IgM (Invitrogen, A21212) and incubated for 45 minutes in the dark at 4° C. with gentle rocking. After secondary antibody binding, cells were washed three times with ice-cold PBS-G, resuspended in 2 ml of ice-cold PBS-G, passed through a 40 micrometer filter and immediately analyzed and sorted on a FACSAria cell sorter (BD Biosciences). For double staining analysis, 1:100 rat anti-human SSEA3 (Millipore, mab4303) and 1:50 mouse anti-human CD105 clone 35 (BD Biosciences, 611314) primary antibodies were used in conjunction with 1:200 DyLight 649-conjugated goat anti-rat (Jackson ImmunoResearch, 112-496-075) and 1:200 FITC-conjugated goat anti-mouse (Invitrogen, M30101) secondary antibodies respectively. The same incubation times and parameters used for single staining analysis were also used for double staining analysis. DAPI (Invitrogen, D1306) was included at a 1:100 ratio for detection and removal of dead cells. In every FACS analysis and purification performed, a control sample of cells (exposed only to secondary antibody) was used to exclude the possibility of non-specific binding and/or autofluorescence. Data were analyzed, DAPI-stained dead cell-exclusion and doublet-exclusion gating was performed and viable single cell subpopulations were sorted using BD FACSDiva Software (BD Biosciences). A bank of HUF1 cells was used to establish SSEA3^{NEGATIVE}, SSEA3^{LOW} and SSEA3^{HIGH} threshold levels for consistent categorization (FIG. 2C) throughout the experiments performed in this study. For sorting purposes, the top 10% of cells, with the highest level of SSEA3 expression, were sorted as representatives for the SSEA3^{HIGH} subpopulation and the bottom 10% of cells with the lowest level of SSEA3 expression were sorted as representatives for the SSEA3^{NEGATIVE} subpopulation. Sorted subpopulations were allowed to adhere for 24 hours before subsequent immunofluorescence, FACS analysis, transcriptional characterization and MSC-differentiation studies were performed.

Global Transcriptional Meta-Analysis

Total RNA was purified from three biological replicates of SSEA3^{HIGH} and SSEA3^{NEGATIVE} FACS-purified populations (24 hours post-sorting) using the RNeasy Mini Kit (Qiagen) according to the manufacturer's instructions. Total RNA was used for U133 plus 2.0 microarray-based global transcriptional analysis using standard Affymetrix protocols (Affymetrix GeneChip Expression Analysis Technical Manual, rev.3. 2001). Affymetrix CEL files for the additional cells and tissues used in this study's meta-analysis were obtained from the Gene Expression Omnibus (GEO, web site ncbi.nlm.nih.gov/geo). The meta-analysis library included two replicates of: adipose tissue-derived mesenchymal stem cells (AT-MSCs, GEO accession numbers GSM501001 and GSM501002 for replicates 1 and 2 respectively), bone marrow-derived mesenchymal stem cells (BM-MSCs, GSM241198 and GSM241200), CD44-expressing epithelial stem cells (GSM379269 and GSM379270), epidermal keratinocytes (GSM173532 and GSM173533), CD200-expressing hair follicle stem cells (GSM538736 and GSM538737), endothelial cells derived from skin microvasculature (GSM700326 and GSM734106) and lymphatic tissues (GSM143717 and GSM143898), skin-derived pericytes (GSM425648 and GSM425649), bone marrow derived hematopoietic stem cells (GSM747521 and GSM747522), bone marrow derived mono-nuclear cells (GSM794774 and GSM794775), human embryonic stem cells (GSM194307 and GSM194308), subcutaneous adipose tissue (GSM646746 and GSM646747), chondrocytes from cartilage (GSM490979 and GSM490980), fetal forebrain tissue (GSM679519 and GSM679522), metaphase II oocytes (GSM304261 and GSM304262) and liver-derived hepatocytes (GSM595063 and GSM595066). Each CEL file was uploaded to GeneSifter (VisX Labs, Seattle, Wash., www.geospiza.com) using the Advanced Upload Method and normalized using the Affymetrix Microarray Analysis Suite (MAS) 5.0 algorithm. Cluster analysis between groups of samples was performed through GeneSifter Project Analysis using analysis of variance (ANOVA) statistical analysis (p<0.01, threshold of 100, Manhattan distance, ward linkage and gene row centering). GeneSifter Pairwise Analysis between samples was performed using all mean normalization and t-test statistical analysis. For each Pairwise Analysis at least two replicates from each cell line or tissue type was compared to a baseline that consisted of three replicates of the SSEA3^{NEGATIVE} cells. Probe sets were considered to be significantly upregulated (compared to the SSEA3^{NEGATIVE} cells) when the p-value (p) was <0.05 and fold change (FC) was equal or greater than 3. For the relative gene expression analysis, the significantly upregulated probe sets (compared to the SSEA3^{NEGATIVE} cells) for each cell and tissue sample in the meta-analysis library was compared to the probe sets upregulated in the SSEA3^{HIGH} cells. The number of upregulated probe sets shared between a sample and SSEA3^{HIGH} cells was compared to the total number of probe sets differentially expressed in the candidate cell or tissue sample and this value was converted to a gene expression ratio based on the average gene expression correlation (value set at 0.0) and the most similar sample analyzed (value set at 1.0). The analysis of variance (ANOVA) statistical analysis and t-test statistical analysis were performed in GeneSifter with p-values set at less than 0.05.

Mesenchymal Stem Cell (MSC) In Vitro Differentiation and Staining

FACS-purified SSEA3-expressing human skin cells were recovered under standard cell culture conditions and allowed to adhere overnight prior to commencement of the MSC-differentiation protocols. Experimental controls were seeded for each MSC differentiation protocol to corroborate the differentiation of each lineage. Specifically, human adipose tissue derived mesenchymal stem cells (AT-MSCs, courtesy of Dr. Mirko Corselli) were included as a positive MSC differentiation control, undifferentiated AT-MSCs were included as a negative control and mixed dermal skin cells (HUF-1 p6) were included in order to assay the presence or absence of MSCs in human skin primary cell cultures prior to flow cytometry based cell sorting. Osteogenesis differentiation was performed using the Stempro Osteogenesis Differentiation Kit as described by the manufacturer (Invitrogen Cat# A10486) except using 5×10² cells/cm². After 21 days of osteogenesis differentiation the level of in vitro mineral deposit was assayed using Alizarin Red S (Sigma Cat# A5533). Adipogenesis differentiation was performed using the Stempro Adipogenesis Differentiation Kit as described by the manufacturer (Invitrogen Cat# A10477) except using 1×10³ cells/cm². After 21 days of adipogenesis differentiation accumulation of intracellular triglycerides was determined with AdipoRed™ Assay Reagent (Lonza Cat# PT-7009). Chondrogenesis differentiation was performed using the Stempro Chondrogenesis Differentiation Kit as described by the manufacturer (Invitrogen Cat# A10582). Specifically, chondrogenic capacity was examined in an optimized 3D micromass culture, as previously described (Johnstone et al. (1998) Exp Cell Res, 238: 265-272), using 2.5×10⁵ cells to form the micromass pellets, and differentiation was induced with the STEMPRO® Chondrogenesis Differentiation Kit (Invitrogen Cat# A10582). Pellets were not disturbed for the first 72 hours and were harvested at day 21, fixed with 4% PFA solution for 30 minutes and embedded with O.C.T (Sakura Cat#4583) and cryosectioned. Thin cryosections (5 μm thick) were mounted and stained for glycoproteins with Toluidine blue O (Sigma Cat#198161) 1% aqueous for 20 minutes at 60° C. followed with three rinses with distilled water and visualization under light microscopy. Differentiation protocols were performed in 5% CO₂ incubators at 37° C., culture media was changed every 2-3 days and all light microscopy based imaging was performed with an AxioCam HR Color Camera using AxioVision Digital Image Processing Software (Axio Observer Inverted Microscope, Carl Zeiss).

Immunofluorescence and Immunohistology

Cultured cells were fixed in 4% paraformaldehyde/PBS for 15 min, washed once with PBS supplemented with 100 mM glycine for 10 min and then washed twice with PBS for 5 min each. Blocking was performed with 4% goat serum in casein-PBS for 1 hr at room temperature. Subsequently 1:100 SSEA3 antibody (Millipore, mab4303) was added to 4% goat serum in casein-PBS and incubated overnight at 4° C. with slow nutation. The next day, cells were washed three times with PBS for 5 min before fluorescent-conjugated secondary Alexa 488-conjugated goat anti-rat IgM (Invitrogen, A21212) was added 1:200 to 4% goat serum in casein-PBS and incubated for 1 hr at room temperature, protected from light. The cells were rinsed with PBS three times and DAPI was used to label the nuclei. A final PBS rinse for 10 min at room temperature was performed. Visualization was performed with an AxioCam MR Monocolor Camera using AxioVision Digital Image Processing Software (Axio Observer Inverted Microscope, Carl Zeiss). Human skin samples were either fixed fresh within 45-60 min of the 4 mm punch skin biopsy (pre-injury samples) or following one month of in vitro biopsy adhesion and culture in DMEM/F12+10% FBS (post-injury samples), the latter of which was used as our in vitro assay for tissue injury. Skin biopsy fragments either pre- or post-injury were cryosectioned (as previously described) and analyzed for SSEA3 expression using the immunofluorescence protocol previously described for in vitro cultured fixed cells.

### Results

SSEA3 Expressing Cells in Human Skin Pre and Post Injury

In vitro adhesion and primary cell derivation was attempted with human skin punch biopsy fragments from three donors. One of the biopsy donors did not demonstrate a successful primary cell culture and was excluded from this study. Immunohistochemical analysis of SSEA3 expression in adult human skin pre-injury revealed that SSEA3 expressing cells were detected in the dermis of human skin and that these cells were extremely rare, representing less than 0.1% of the cells in the adult dermal tissue pre-injury. However, consistent with the in vitro model for dermal tissue injury and regeneration, a large increase in the number of SSEA3 expressing cells was observed with a greater than 20-fold local increase in the number of SSEA3 positive cells following injury. The SSEA3 expressing cells appeared as tightly compacted spheres of SSEA3 positive cells with more dispersed SSEA3 positive cells migrating out from this sphere. These SSEA3 expressing spheres were observed in multiple biological replicates. This result indicates that SSEA3 expressing cells in human skin dermis are linked to the skin injury/regeneration response.

Global Transcriptional Meta-Analysis

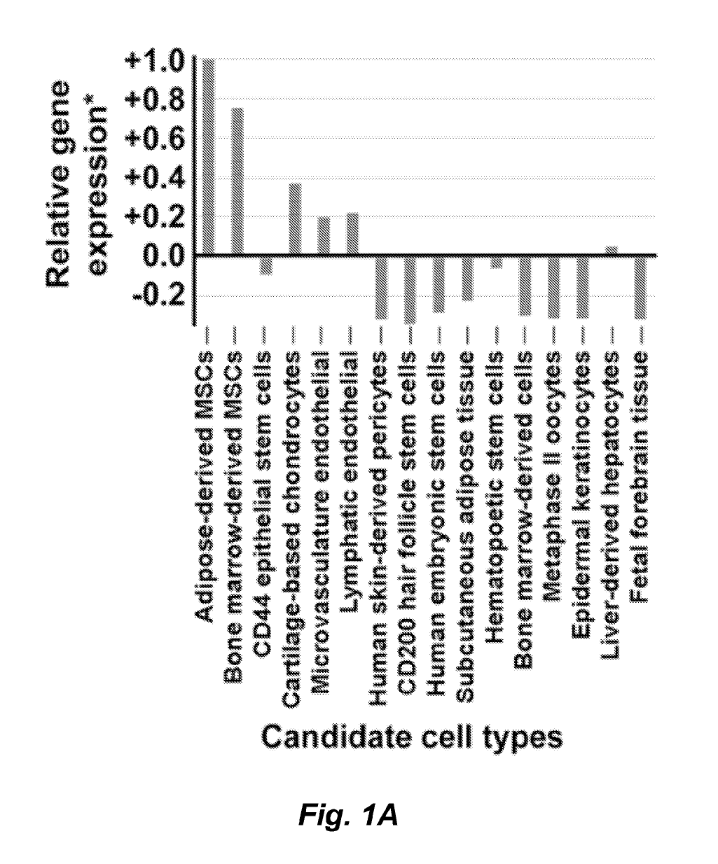

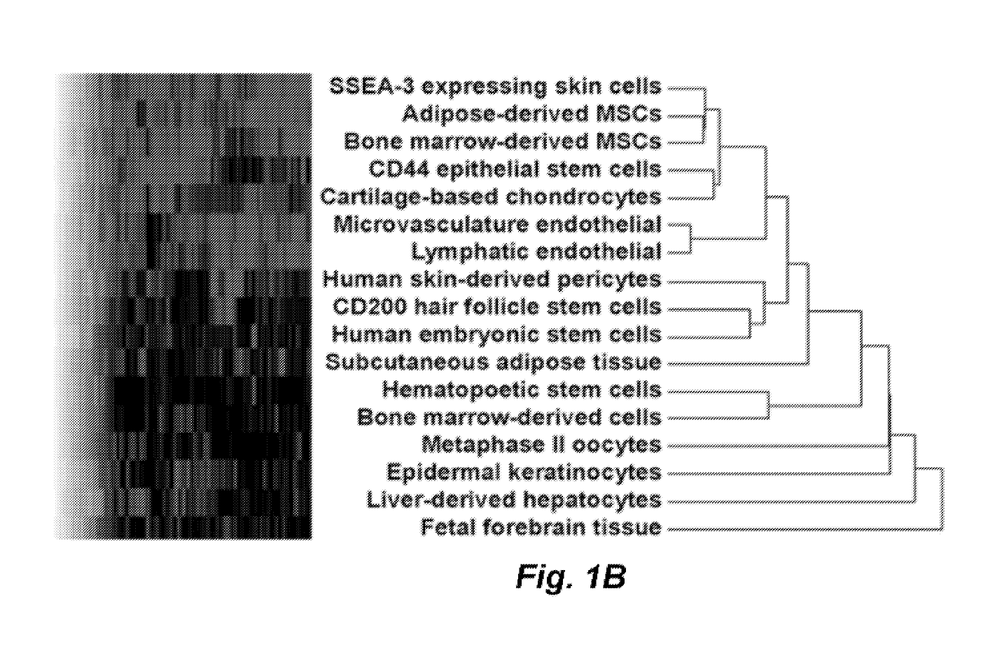

Global transcriptional meta-analysis was performed using the following cell and tissue samples in our meta-analysis library: low passage (p3) SSEA3^{HIGH} skin-derived human somatic cells 24 hours after FACS-based purification, adipose tissue-derived mesenchymal stem cells (AT-MSCs), bone marrow-derived mesenchymal stem cells (BM-MSCs), CD44-expressing epithelial stem cells, epidermal keratinocytes, CD200-expressing hair follicle stem cells, endothelial cells derived from skin microvasculature and lymphatic tissues, skin-derived pericytes, bone marrow derived hematopoietic stem cells, bone marrow derived mono-nuclear cells, embryonic stem cells, subcutaneous adipose tissue, chondrocytes from cartilage, fetal forebrain tissue, metaphase II oocytes and liver-derived hepatocytes. Pairwise analysis of the SSEA3^{HIGH} subpopulation against the SSEA3^{NEGATIVE} subpopulation revealed over 200 significantly upregulated (p<0.05, fold change >3) probe sets (Gene Expression Omnibus, GEO, web site ncbi.nlm.nih.gov/geo #GSE33066). Pairwise analysis of the other members of the meta-analysis library was performed (compared to the SSEA3^{NEGATIVE} cells) and the significantly upregulated probe sets (p-value <0.05, fold change >3) were compared to the previously identified SSEA3^{HIGH}-specific probe sets. This focused gene expression analysis revealed that the SSEA3^{HIGH} cells were more transcriptionally similar to adipose-tissue derived MSCs, closely followed by bone marrow derived MSCs (FIG. 1A). However, unbiased cluster analysis also revealed that the adipose tissue and bone marrow derived MSCs were transcriptionally closer to each other than to SSEA3-expressing cells (FIG. 1B), highlighting that the SSEA3-expressing human skin cells identify a non-MSC subpopulation.

Flow Cytometric Analysis and Purification of Primary Human Skin Cells

SSEA3-expressing human skin cells co-express the endoglin (CD105) epitope have been detected using antibody CD105 clone 266 (BD Bioscience, 560839) (Kuroda et al. (2010) Proc. Natl. Acad. Sci. USA, 107: 8639-8643; Wakao et al. (2011) Proc. Natl. Acad. Sci. USA, 108: 9875-9880). These SSEA3/CD105 clone 266 identified cells are referred to as multipotent stress enduring (MUSE) cells in reference to their multilineage differentiation capacity (Kuroda et al. (2010) supra; Wakao et al. (2011) supra). However, prior work examined the co-expression dynamics of SSEA3 and CD105 clone 35 (BD Biosciences, 611314). The percent of antibody bound cells was investigated using either CD105 antibody clones 266 and 35. As previously reported, CD105 clone 266 bound to almost 100% of primary cultured human skin cells (Kuroda et al. (2010) supra; Wakao et al. (2011) supra) (FIG. 2A, left panel). However, CD105 clone 35 (CD105c35) bound to only around 1% primary cultured human skin cells (FIG. 2A, right panel). It is considered that CD105c35 is binding a rarer variant of endoglin (CD105) while the clone 266 antibody is binding an endoglin variant ubiquitously expressed on human primary skin cells. Regardless, CD105c35 accurately identifies a useful subpopulation of skin cells. Interestingly, CD105c35 only bound to about 1% of bone marrow derived MSCs, suggesting that this antibody is not an MSC marker.

Next, flow cytometric analysis was performed using both SSEA3 antibody and CD105c35 antibody. It was observed that there is a strong correlation between the cells that expressed the highest levels of SSEA3 and the cells that expressed CD105c35 (FIG. 2B). Approximately 1% of adult human skin-derived cells expressed both SSEA3 and CD105c35 (FIG. 2B). Next, flow cytometry was used to examine the dynamic expression of SSEA3 on primary human skin cells (using the HUF1 line previously described (Byrne et al. 2009)). It was first verified, as previously reported, that within the primary HUF1 skin culture, cells expressed SSEA3 heterogeneously (Byrne et al. (2009) supra) with 54% of the cells demonstrating no significant SSEA3 expression (SSEA3^{NEGATIVE}), 33% demonstrating low SSEA3 expression (SSEA3^{LOW}) and 13% of the skin cells demonstrating high SSEA3 expression (SSEA3^{HIGH}, FIG. 2C). Fluorescence activated cell sorting (FACS) was then used to purify representatives of the SSEA3^{NEGATIVE} and SSEA3^{HIGH} subpopulations (FIG. 2C) from HUF1 cells that had been derived from the human skin biopsy fragments and maintained in culture for five passages (p5). Subpopulations were cultured under standard fibroblast culture conditions and then re-examined at intervals to assess dynamics of SSEA3 expression. One day post-FACS, 100% of the cells in the SSEA3^{NEGATIVE}-sorted subpopulation maintained their SSEA3^{NEGATIVE} phenotype; notably, however, while after one week in culture 83% of the cells in the SSEA3^{NEGATIVE}-sorted subpopulation still maintained their SSEA3^{NEGATIVE} phenotype, 12% were now SSEA3^{LOW} and 5% were SSEA3^{HIGH} (FIG. 2C). Even more markedly, one day after FACS-based purification, only 43% of the SSEA3^{HIGH}-sorted subpopulation maintained the SSEA3^{HIGH} phenotype and after one week in culture only 18% were still considered SSEA3^{HIGH}, 32% were SSEA3^{LOW} and 50% were SSEA3^{NEGATIVE}. This evidence suggests that expression of the SSEA3 epitope is dynamic in cell culture. No significant difference in the rate of proliferation from the SSEA3^{HIGH}-sorted and SSEA3^{NEGATIVE}-sorted populations was observed, with population doubling times of 36.0+/−1 hr and 36.7+/−1.4 hr respectively. Immunocytochemical analysis of the SSEA3^{NEGATIVE}-sorted and SSEA3^{HIGH}-sorted subpopulations confirmed heterogeneity of SSEA3 expression in both subpopulations following several days of cell culture. Dynamic expression of the SSEA3 molecule in vitro might reflect a culture induced instability. Specifically, expression of SSEA3 on the cell surface may be more stable in skin and may be destabilized over time as cells migrate from their original niche in the skin. To test this, the SSEA3^{HIGH} subpopulations from both higher passage (p8) and lower passage (p3) HUF1 cells were FACS-purified and analyzed. Higher passage (p8) HUF1 cells had been in culture approximately one month longer than lower passage (p3) HUF1 cells. The higher passage (p8) HUF1 cells quickly lost SSEA3 expression, with less than 10% of these cells still demonstrating an SSEA3^{HIGH} phenotype after one week (FIG. 2D). In comparison, the lower passage (p3) HUF1 cells demonstrated significantly increased stability of SSEA3 expression, with nearly half of the low passage cells maintaining an SSEA3^{HIGH} phenotype after one week (FIG. 2D).

Mesenchymal Stem Cell (MSC) Analysis in Adult Human Skin Primary Cultures

Human skin cells that co-expressed both SSEA3 and CD105c35 (referred to here as SERA cells) were purified using FACS and analyzed for MSC differentiation potential alongside human adipose tissue derived MSCs (AT-MSCs, included as a positive MSC differentiation control) and mixed dermal skin cells (HUF-1 p6, included to assay the presence or absence of MSCs in human skin primary cell cultures prior to flow cytometry based cell sorting). After 21 days of osteogenesis, adipogenesis or chondrogenesis differentiation, cells were assayed for formation of osteoblasts (observed via Alizarin Red S staining), adipocytes (observed via AdipoRed staining) or chondrocytes (observed via Toluidine blue O staining) under their respective culture conditions. The AT-MSC positive control cells demonstrated successful differentiation into osteoblasts, adipocytes and chondrocytes demonstrating the success of the MSC differentiation protocols utilized here. While a rare (<1%) functional MSC subpopulation was observed in human skin primary cultured cells that was capable of differentiating into osteoblasts, adipocytes and chondrocytes the SERA cells did not demonstrate MSC differentiation capacity suggesting that the SERA cells and MSCs represent two different rare subpopulations in human dermis.

### Discussion

This study confirms previous reports that identified a population of SSEA3 expressing cells within and derived from adult human skin (Byrne et al. (2009) supra; Kuroda et al. (2010) supra; Wakao et al. (2011) supra), and further identified a rare subpopulation. The response of these SSEA3 expressing skin cells to tissue injury was examined. In vitro biopsy adhesion, cell migration and extended culture (for 1 month) were used as an in vitro assay for tissue injury. This in vitro assay for tissue injury was based on the premise that cells on the periphery of newly damaged dermal tissue (whether in vivo or in vitro) would have the same basic responses to dermal tissue damage.

This premise is consistent with research that has demonstrated similarities in the in vivo and in vitro response of skin cells to tissue damage following thermally-induced injury (Coolen et al. (2008) Wound Repair Regen, 16: 559-567). Immunohistochemical analysis revealed that the SSEA3 expressing cells are extremely rare in normal human skin, representing less than 0.1% of the cells in the pre-injury skin. Following the in vitro injury assay at least a 20-fold local increase in the number of SSEA3 positive cells was observed and SSEA3 expressing cells appeared to be migrating away from central spheres of tightly packed SSEA3 expressing cells. Although the function of these tightly packed SSEA3 expressing spheres was not certain, the present study indicates that they play a role in the regeneration of human skin in response to injury. These data provide empirical support that SSEA3 expression significantly correlates with tissue regeneration in human skin.

These SERA cells were derived through primary cell culture, purified by FACS and characterized at multiple time points over extended in vitro culture. It was observed that the longer the primary skin cells were cultured in vitro, the lower the SSEA3 expression stability following FACS-based purification, with cells cultured an extra month in vitro demonstrating a five-fold decrease in their SSEA3 expression stability one week after FACS-based purification. This result supports that the stability of SSEA3 expression is negatively impacted by extended in vitro culture. Whether optimization of in vitro conditions might stabilize SSEA3 expression over extended culture is yet to be determined.

Research by Dezawa and colleagues (Wakao et al. (2011) Proc. Natl. Acad. Sci. USA, 108: 9875-9880) has demonstrated that skin-derived SSEA3 expressing cells represent a different population from previously identified ASC-like subpopulations in the skin, such as skin-derived precursor (SKP) cells (Fernandes et al. (2004) Nat Cell Biol, 6: 1082-1093; Biernaskie et al. (2009) Cell Stem Cell, 5: 610-623; Toma et al. (2001) Nat Cell Biol, 3: 778-784), melanocyte stem-cells (Nishimura et al. (2002) Nature, 416: 854-860), endothelial precursors (Middleton et al. (2005) J Pathol, 206: 260-268), pericytes (Crisan et al. (2008) Cell Stem Cell, 3: 301-313) or neural crest-derived stem cells (Nagoshi et al. (2008) Cell Stem Cell, 2: 392-403). The results in this study demonstrated that the SSEA3 expressing cells derived in this example shared a closer transcriptional profile with MSCs derived from adipose tissue and bone marrow, than with CD200 expressing hair follicle stem cells (Garza et al. (2011) J Clin Invest, 121: 613-622), CD44 expressing epithelial stem cells (Bhat-Nakshatri et al. (2010) BMC Cancer, 10: 411), skin-derived endothelial cells (Kanki et al. (2011) EMBO J, 30: 2582-2595) or skin-derived pericytes (Paquet-Fifield et al. (2009) J Clin Invest, 119: 2795-2806), indicating that these SSEA3 expressing cells identify an ASC-like population in human skin.

However, while a rare subpopulation of functional MSCs was observed in human skin primary cultures, the SERA cells did not demonstrate MSC differentiation capacity, indicating that the SERA cells and MSCs represent two different subpopulations in human skin, possibly with different and/or complimentary roles in vivo. While not a canonical MSC marker, it remains possible that selection for the SSEA3 biomarker may still provide a limited degree of enhancement towards the adipogenic pathway, similar to that previously observed for SSEA4 (Vaculik et al. (2012) J Invest Dermatol, 132: 563-574). Regarding the functional skin-derived MSCs observed in this study, it was clear which biomarker profile would facilitate rapid identification, isolation and expansion of these rare cells from adult human skin biopsy primary cultures, however the top MSC candidate biomarkers include CD10, CD13, CD26, CD29, CD34, CD44, CD54, CD71, CD73, CD90, CD105 (clone 266), CD106, CD146, CD166, CD271, ITGA11, STRO-1 and SSEA4 (Vaculik et al. (2012) supra; Mafi et al. (2011) Open Orthop J, 5: 253-260; Halfon et al. (2011) Stem Cells Dev, 20: 53-66). As demonstrated in Example 2 and as disclosed herein, the markers CD146 and CD271 provides identification of a particularly useful subpopulations of MSCs. The derivation of MSCs from skin punch biopsies, which are quick, simple and minimally invasive to perform, may be preferable for many patients to current MSC derivation protocols that require the surgical aspiration of adipose tissue and/or bone marrow. The identification, efficient purification and large-scale expansion of these rare subpopulations (SERA cells and MSCs) from heterogeneous adult human skin primary cell cultures have applications for patient-specific cellular therapies.

### Example 2

### Isolation and Differentiation of Skin-Derived CD146 and CD271-Mesenchymal Stem Cells

Mesenchymal stem cells (MSCs) observed in human skin cell cultures have applications for storage, diagnostics, spinal fusion, bone and joint repair and potential treatment of other degenerative diseases. A new form of mesenchymal stem cells (MSCs) has been discovered as a rare subpopulation of primary human skin cell cultures. This subpopulation is characterized by the cell surface biomarker cluster of differentiation (CD) 146 and CD271. It has been discovered that differentiated mesenchymal stem cells can be produced by purifying CD146 and CD271-expressing cells from primary human cell cultures. These purified CD146 and CD271-expressing cells can be differentiated by, for example, growing to confluence and differentiating in optimized osteogenesis differentiation media (Lonza, hMSC osteogenic differentiation BulletKit™, Cat. No. PT-3002). Doing so resulted in relatively pure populations of Human Osteogenic Dermal Mesenchymal Stem Cells (HOD-MSCs). The protocol for the culture and analysis of HOD-MSCs was performed as described in Example 1 except modified by using the aforementioned optimizations; specifically, CD146- and CD271-based purifications and optimized cell culture and differentiation media.

These HOD-MSCs can have applications for patient-specific osteogenesis-based therapeutics, such as for critical bone loss and spinal fusion. Cells will typically be used as a solution, with cells suspended in either Phosphate Buffered Saline (PBS) or cell culture medium. Large numbers of HOD-MSCs (10⁸-10⁹ or more) can be grown in culture for uses that required it. For example, large numbers of HOD-MSCs are useful to form a large amount of bone in vivo. As another example, HOD-MSCs can be used to augment iliac autograft based spinal fusion.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the materials for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different cell types does not indicate that the listed cell types are obvious one to the other, nor is it an admission of equivalence or obviousness.

Those skilled in the art will recognize, or be able to ascertain using no more than routine experimentation, many equivalents to the specific embodiments of the method and compositions described herein. Such equivalents are intended to be encompassed by the following claims.

1. An isolated population of human mesenchymal stem cells (MSCs) derived from the dermis of human expressing cluster of differentiation 271 (CD271) (CD271-MSC).

2. The population of claim 1, wherein the population contains less than 5% other cell types in the tissue from which the cells were derived that do not significantly express CD271.

3. The population of claim 1, wherein the population is a population of human mesenchymal stem cells (MSCs) derived from the dermis of human expressing cluster of differentiation 146 (CD146) (CD146-MSCs) and CD271 (CD271-MSCs).

4. The population of claim 3, wherein the population contains less than 5% other cell types in the tissue from which the cells were derived that do not significantly express CD146 and CD271.

5. The population of claim 1, wherein the cells differentiate into adipocytes, osteoblasts, or chondrocytes under conditions that induce such differentiation.

6. The population of claim 1, wherein the cells express one or more markers selected from the group consisting of CD10, CD13, CD26, CD29, CD34, CD44, CD54, CD71, CD73, CD90, CD105 (clone 266), CD106, CD166, ITGA11, STRO-1 and SSEA4.

7-11. (canceled)

12. A cell line or cell strain comprising a plurality of cells proliferated ex vivo from the population of claim 1.

13. A cell line or cell strain comprising a plurality of cells differentiated ex vivo from the population of claim 1.

14. A cell line or cell strain comprising a plurality of induced pluripotent stem cells (iPSCs) derived ex vivo from the population of claim 1.

15. A therapeutic composition comprising the population according to claim 1 and a pharmaceutically acceptable platform.

16. The composition of claim 15, wherein the platform is a matrix, a scaffold, a graft, an implant, a solid support, a hydrogel, a polymer, a medium, a gel, a slurry, a device, or a combination.

17. The composition of claim 15, wherein the platform is a graft, wherein the graft comprises a scaffold and a therapeutic component, wherein the scaffold comprises a porous matrix of fibers forming interstices, wherein the therapeutic component comprises a cellular component and a non-cellular component, and wherein the cellular component comprises the cell or cell line or cell strain.

18. The composition of claim 17, wherein the cellular component comprises the SERA cells or cells derived from the MSCs.

19. The composition of claim 18, wherein the cells derived from the MSCs are selected from the group consisting of neural cells and cardiac cells.

20. The composition of claim 18, wherein the non-cellular component comprises one or more pharmaceutically active agents selected from the group consisting of growth factors, anti-inflammatories, antibiotics, and antivirals.

21. The composition of claim 15, wherein the platform is a skin filler, wherein the population is a population of SERA cells.

22. A method of treating a mammal, the method comprising applying or administering to the mammal (a) CD146-MSCs of claim 1 and/or CD271-MSCs.

23. A method of treating a mammal, the method comprising applying or administering to the mammal a cell line or cell strain of claim 12.

24. A method of treating a mammal, the method comprising applying or administering to the mammal a therapeutic composition of claim 15.

25. A method of producing isolated CD146-MSCs and/or CD271-MSCs, the method comprising:
culturing cells from a tissue sample comprising dermis of human skin; and
isolating a subpopulation of the cultured cells that express CD 146 or CD271.

26-27. (canceled)

28. A kit comprising antibodies specific for detection and sorting of cells expressing SSEA3 and CD105 (clone 35) CD146 or CD271.

29-30. (canceled)
